SEQUENCE LISTING (18 sequences)

SEQ ID NO: 1 - DNA, 2732 bp, Homo sapiens
  ref <308>: DATABASE ACCESSION NUMBER: GenBank / X05199
  ref <309>: DATABASE ENTRY DATE: 1995-03-21
  ref <313>: RELEVANT RESIDUES: (1)..(2732)
aacaacatcctgggattgggacccactttctgggcactgctggccagtcccaaaatggaacataaggaagtggttcttctacttcttttatttctgaaatcaggtcaaggagagcctctggatgactatgtgaatacccagggggcttcactgttcagtgtcactaagaagcagctgggagcaggaagtatagaagaatgtgcagcaaaatgtgaggaggacgaagaattcacctgcagggcattccaatatcacagtaaagagcaacaatgtgtgataatggctgaaaacaggaagtcctccataatcattaggatgagagatgtagttttatttgaaaagaaagtgtatctctcagagtgcaagactgggaatggaaagaactacagagggacgatgtccaaaacaaaaaatggcatcacctgtcaaaaatggagttccacttctccccacagacctagattctcacctgctacacacccctcagagggactggaggagaactactgcaggaatccagacaacgatccgcaggggccctggtgctatactactgatccagaaaagagatatgactactgcgacattcttgagtgtgaagaggaatgtatgcattgcagtggagaaaactatgacggcaaaatttccaagaccatgtctggactggaatgccaggcctgggactctcagagcccacacgctcatggatacattccttccaaatttccaaacaagaacctgaagaagaattactgtcgtaaccccgatagggagctgcggccttggtgtttcaccaccgaccccaacaagcgctgggaactttgcgacatcccccgctgcacaacacctccaccatcttctggtcccacctaccagtgtctgaagggaacaggtgaaaactatcgcgggaatgtggctgttaccgtttccgggcacacctgtcagcactggagtgcacagacccctcacacacataacaggacaccagaaaacttcccctgcaaaaatttggatgaaaactactgccgcaatcctgacggaaaaagggccccatggtgccatacaaccaacagccaagtgcggtgggagtactgtaagataccgtcctgtgactcctccccagtatccacggaacaattggctcccacagcaccacctgagctaacccctgtggtccaggactgctaccatggtgatggacagagctaccgaggcacatcctccaccaccaccacaggaaagaagtgtcagtcttggtcatctatgacaccacaccggcaccagaagaccccagaaaactacccaaatgctggcctgacaatgaactactgcaggaatccagatgccgataaaggcccctggtgttttaccacagaccccagcgtcaggtgggagtactgcaacctgaaaaaatgctcaggaacagaagcgagtgttgtagcacctccgcctgttgtcctgcttccagatgtagagactccttccgaagaagactgtatgtttgggaatgggaaaggataccgaggcaagagggcgaccactgttactgggacgccatgccaggactgggctgcccaggagccccatagacacagcattttcactccagagacaaatccacgggcgggtctggaaaaaaattactgccgtaaccctgatggtgatgtaggtggtccctggtgctacacgacaaatccaagaaaactttacgactactgtgatgtccctcagtgtgcggccccttcatttgattgtgggaagcctcaagtggagccgaagaaatgtcctggaagggttgtgggggggtgtgtggcccacccacattcctggccctggcaagtcagtcttagaacaaggtttggaatgcacttctgtggaggcaccttgatatccccagagtgggtgttgactgctgcccactgcttggagaagtccccaaggccttcatcctacaaggtcatcctgggtgcacaccaagaagtgaatctcgaaccgcatgttcaggaaatagaagtgtctaggctgttcttggagcccacacgaaaagatattgccttgctaaagctaagcagtcctgccgtcatcactgacaaagtaatcccagcttgtctgccatccccaaattatgtggtcgctgaccggaccgaatgtttcatcactggctggggagaaacccaaggtacttttggagctggccttctcaaggaagcccagctccctgtgattgagaataaagtgtgcaatcgctatgagtttctgaatggaagagtccaatccaccgaactctgtgctgggcatttggccggaggcactgacagttgccagggtgacagtggaggtcctctggtttgcttcgagaaggacaaatacattttacaaggagtcacttcttggggtcttggctgtgcacgccccaataagcctggtgtctatgttcgtgtttcaaggtttgttacttggattgagggagtgatgagaaataattaattggacgggagacagagtgacgcactgactcacctagaggctgggacgtgggtagggatttagcatgctggaaataactggcagtaatcaaacgaagacactgtccccagctaccagctacgccaaacctcggcattttttgtgttattttctgactgctggattctgtagtaaggtgacatagctatgacatttgttaaaaataaactctgtacttaactttgatttgagtaaattttggtttt

SEQ ID NO: 2 - protein, 810 aa, Homo sapiens
  ref <308>: DATABASE ACCESSION NUMBER: GenBank / P00747
  ref <309>: DATABASE ENTRY DATE: 1986-07-21
  ref <313>: RELEVANT RESIDUES: (1)..(810)
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 3 - DNA, 7258 bp, Homo sapiens
  ref <308>: DATABASE ACCESSION NUMBER: GenBank / X02419
  ref <309>: DATABASE ENTRY DATE: 1997-02-07
  ref <313>: RELEVANT RESIDUES: (1)..(7258)
ttcaataggaagcaccaacagtttatgccctaggactttgttcccacaatcctgtaacatcatatcacgacacctaacccaatccttatcaagccctgtcaaaaacggactttaaaccaagctgcaaattttcagtaatctggccttgcctttccccctctgatagcaccatcaaacaaacccccttactgccgaaagcaataagcccggctttgttccatccactggttgtgttggtgatatctggggactgccactgaacagacgcacagagggagcccctacaggcaggggtttttctgtctgtgcttcttgggagagtatgtctcgtacatttgtcgcgtgatgaagacttcacagctccatccagcgaccagactcacagctccatccagctgcggcaagggggtctgaggcagtcttaggcaagttggggcccagcgggagaagttgcagaagaactgattagaggacccaggaggcttcagagctgggctgaggtagagagtctcctgtgcgccttctctcctctctgcaattcggggactccttgcactggggcaggccccggcaggtgcatgggaggaagcacggagaatttacaagcctctcgattcctcagtccagacgctgttgggtcccctccgctggagatcgcgcttcccccaaatctttgtgagcgttgcggaagcacgcggggtccgggtcgctgagcgctgcaagacaggggagggagccgggcgggagagggaggggcggcgccggggcgggccctgatatagagcaggcgccgcgggtcgcagcacagtcggagaccgcagcccggagcccgggccagggtccacctgtccccgcagcgccggctcgcgccctcctgccgcagccaccggtgagtgccgcggtcctgagatccccgggccggatgcgcggcggccccagctcccgagcgtctgcctgccccgccctgggctgcccgggctccctgggctccccggcggctgcacggagtcaaggcgccccgtcccgggcgtcccccgcgggtgccgatccaggctgcccggagtccggagcccatagaggagagagacagctggggagcctggtcaccgcgggcatctcccctgcgctgcagtcgcccgcctggcctgccttcccgttcctccgcctcttgccctgacttctccttcctttgcagagccgccgtctagcgccccgacctcgccaccatgagagccctgctggcgcgcctgcttctctgcgtcctggtcgtgagcgactccaaagtgagtgcgctcttgctttgactgatgctgcccaaggacctctgatcagcaccaggggagaggaggggctgctcagggagctggggtctccggattccatccacagcagggccagactctccccaggaaatgggacagggtggcagcggaggcttgagaaccacgggggttggcactggctggcaagggaggaagagggccaccgggactgccccagcctgcgggcatctggtagatgaagcttaatccatttctcctggctggaaaccatggtcttccatttgagaactagatacgaacagggtgaggcgagagggagagggaagagtgggttttgggattggggccagtttaccctcaccctggatccctggagcatgggacctttgatgaagcctcctcccgaatctcttccagggcagcaatgaacttcatcaagttccatgtgagtatccacccctacaacagttggctgcacagacaagttgggaaggcttcaggggacactcccctccctgccctctgctgcagcgtgcgccaccccttaccacttccactccccctcgcttaccccacctttgttctctccagcgaactgtgactgtctaaatggaggaacatgtgtgtccaacaagtacttctccaacattcactggtgcaactgcccaaagaaattcggagggcagcactgtgaaataggtatggggatctccactgcaactgggagagaaatttggggacagggagggatgggtgggaggcaagagcaggcaggagttaggagctggaggtagggtgggtgacatcttcatccctatgtgacaagcataaacacacacacacgctcacgaaacagtggccacacaaatgtgaggtggggttggaaggagaccctgtccagtcttctggcaggtctgaaacgacatctttaaaatgtccgttggcagccgggcatggtggctcacgcttgtaatcccagcattttgagaggtcaagtttgagtggatcatttaggtcaggagttcaagaccagcctggacaacatggtgtaaccctgcctctactaaaaatgcaaaaatcagcctggcatggtggtggatgcctgtagtcccagctacttgggaggctgaggcaggagaattgcttgaacatgggaggccagatctcagtgagctgagatcacaccactgcactccaactgggcgacagagcaagactccatctcaaaaaaaaaaaaaaataaaagttagttggaatgttcttctctttctcatattctctcatcctcctgtccccttgtagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggtgaggtgggggcaacaaggaccaaaagccctccctacagcttcccagaaaccttgttaccatccccttctcccagagggctggccatagcacaagagaagtgcggcctctggttgagtcttccctgaggggaggaggcagggaaggccctctgggttggaatgacatcccctatctttctgtgttgtgccaggaacccagacaaccggaggcgaccctggtgctatgtgcaggtgggcctaaagccgcttgtccaagagtgcatggtgcatgactgcgcagatggtgagcatcactgacctgctgatgacaggtgggtggaaggggacaaacttacatgtccccttattccatcacaggaggactgaggaggtggggggtgcccgagagggatgctttctcctacctgcctccctaagacatccctctgtttgtcctccaggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatgagcccttgctgggtgatcagcgccacacactgcttcatgtacggccctgggtttctcctcttcgactcttctgccccaccccaagcacatccctttctccttcccagcaaagtgttccgcctcatttctccctcatctgcccctgtccatgcgcccatggccttggggacaagtcgtgctttgaggcctctagggagggaaggaagaagtggcatgatttcatgggactaagctgtttgatgggtatcttcttccacagtgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattggtgagggggaacgcccgcgactactgtggccataatggcttggggagagtgggacccagggagagactggagctgagttgaagctgccggtggggcaggggtggggcgagggaccttgaagcctcgatatacatgacaaaggatggcagggaagagttccatgaagtctgaggggcctggtgctcctctggagagaccctgaatttccccaacaagtagccctcttgcgagtggaaacagccctgtgggtatatggcttgggctgggaaggccctgtttatatgaattagaaaaagacacaccttcctttgtgggatgcagcctctgtctgtgctaggatatagaacttggagaatggagccttgggatggattccagcctaactacctcagggggatcctctagagtgcagctgggagtttttgcagaaacgacctgtacagctgtatgcagtggctctggccatccaagcctttttcaacacctggaacaaagcccttggggcatggggcaggggaggtttccaggtgataagcgaccagcagacctccctggatgactgacctagggataggcatagctacttcctcggcacttggaggggacagatggggaccgcctaaccagtagtgatctttctcctctgaccctctgtcctcccccagccttgctgaagatccgttccaaggagggcaggtgtgcgcagccatcccggactatacagaccatctgcctgccctcgatgtataacgatccccagtttggcacaagctgtgagatcactggctttggaaaagagaattctagtaagtgacaattgcgactgacttagaaggtcctgaggagtgttttgacctgaaaatgagcccagtgtgatcaagggaagactgcagagttagaggtgggagcactgaggcggtggcagatgggtccagggatggatgaagagtgttgtttagggagcgatgggctgcaaaggtaaatagatggtaggggctataggtggaggtaaatggctcagatttgcatggagagagaataatgggcctctccctgggtgatgatactttatggtgtcccctctctggcgagacgtcccacgtggaggcagataaatcttgatgcaaacgcctccctgttttctccacctagccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccaggtgagtgttccaagcatctctctccacctcttccatatctccccagagctcctgggcttgttccagccagcttaagggtgtctctctctagccaaagccctaagtagccagaatcaggagctcaggtctttgagggtttaaaccagtccttatgtgtttgccagacattaccaaaaaaatcccagctctgcgctagtcacttcagactgggggcacgagatcctagaaagaggaaacagtaaaagacaatgtaactcagtgcccagggtgtgttgtgaactataaatgatcaggtgttcaggagagggaggtgagtgccaacctgagggtcagggaggggaggctttaaaggaaatgtgacttgataggcatttgaagaggcagagggaagaaaggaaggtgtttcagttgaaagatacaaaactgagaaggaggctggcatattccgggtggggaggagaactagggtctgggagtgtggatggaatagtggcagatgacagggcttttaaagccaagcaggggattttccaacttcgatgtggtagaaatggggctgcgtcaggcacagtggctcatgcctgtaatcccagcattgggctaggccgtagtcgatggatcattgaggccagagttgagaccggcctggaccaacatggtgaaaccctgtgtctactaaaaaatgcaaaaaaaaaaattagccaggtgtggtggtgcctgcctgtaatcccagctaatcaggaggctgagacatggaatcgcttgagcacaggaggcaagtttgacgtgagctgagatcacgtcattgcacgccagcctgggcgacagagcgagattctgtcctcccgccgaaaaaagaaagaaaatgggaagtcgctaaggactttgactgggaaactcttccctctctctggtatggttgggtgatgggatcagaaatcccctcctcacttctctagggctcatcttttgtatctttggcgtcacagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctgagggtccccagggaggaaacgggcaccacccgctttcttgctggttgtcatttttgcagtagagtcatctccatcagctgtaagaagagactgggaagataggctctgcacagatggatttgcctgtgccacccaccagggtgaacgacaatagctttaccctcaggcataggcctgggtgctggctgcccagacccctctggccaggatggaggggtggtcctgactcaacatgttactgaccagcaacttgtctttttctggactgaagcctgcaggagttaaaaagggcagggcatctcctgtgcatgggtgaagggagagccagctcccccgacggtgggcatttgtgaggcccatggttgagaaatgaataatttcccaattaggaagtgtaacagctgaggtctcttgagggagcttagccaatgtgggagcagcggtttggggagcagagacactaacgacttcagggcagggctctgatattccatgaatgtatcaggaaatatatatgtgtgtgtatgtttgcacacttgtgtgtgggctgtgagtgtaagtgtgagtaagagctggtgtctgattgttaagtctaaatatttccttaaactgtgtggactgtgatgccacacagagtggtctttctggagaggttataggtcactcctggggcctcttgggtcccccacgtgacagtgcctgggaatgtacttattctgcagcatgacctgtgaccagcactgtctcagtttcactttcacatagatgtccctttcttggccagttatcccttccttttagcctagttcatccaatcctcactgggtggggtgaggaccactccttacactgaatatttatatttcactatttttatttatatttttgtaattttaaataaaagtgatcaataaaatgtgatttttctgatgacaaatctccctggtgcttgtatgggaaggagttggagtacataaaaaggagaaaataacaaaggtgg

SEQ ID NO: 4 - protein, 431 aa, Homo sapiens [sequence omitted]

SEQ ID NO: 5 - DNA, 1363 bp, Homo sapiens
  ref <308>: DATABASE ACCESSION NUMBER: GenBank / X51675
  ref <309>: DATABASE ENTRY DATE: 1992-12-17
  ref <313>: RELEVANT RESIDUES: (1)..(1363)
agagaagacgtgcagggaccccgcgcacaggagctgccctcgcgacatgggtcacccgccgctgctgccgctgctgctgctgctccacacctgcgtcccagcctcttggggcctgcggtgcatgcagtgtaagaccaacggggattgccgtgtggaagagtgcgccctgggacaggacctctgcaggaccacgatcgtgcgcttgtgggaagaaggagaagagctggagctggtggagaaaagctgtacccactcagagaagaccaacaggaccctgagctatcggactggcttgaagatcaccagccttaccgaggttgtgtgtgggttagacttgtgcaaccagggcaactctggccgggctgtcacctattcccgaagccgttacctcgaatgcatttcctgtggctcatcagacatgagctgtgagaggggccggcaccagagcctgcagtgccgcagccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccgaggccccatgaatcaatgtctggtagccaccggcactcacgaaccgaaaaaccaaagctatatggtaagaggctgtgcaaccgcctcaatgtgccaacatgcccacctgggtgacgccttcagcatgaaccacattgatgtctcctgctgtactaaaagtggctgtaaccacccagacctggatgtccagtaccgcagtggggctgctcctcagcctggccctgcccatctcagcctcaccatcaccctgctaatgactgccagactgtggggaggcactctcctctggacctaaacctgaaatccccctctctgccctggctggatccgggggacccctttgcccttccctcggctcccagccctacagacttgctgtgtgacctcaggccagtgtgccgacctctctgggcctcagttttcccagctatgaaaacagctatctcacaaagttgtgtgaagcagaagagaaaagctggaggaaggccgtgggcaatgggagagctcttgttattattaatattgttgccgctgttgtgttgttgttattaattaatattcatattatttattttatacttacataaagattttgtaccagtgg

SEQ ID NO: 6 - protein, 335 aa, Homo sapiens
  ref <308>: DATABASE ACCESSION NUMBER: GenBank / Q03405
  ref <309>: DATABASE ENTRY DATE: 1994-02-01
  ref <313>: RELEVANT RESIDUES: (1)..(335)
MGHPPLLPLLLLLHTCVPASWGLRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYRSGAAPQPGPAHLSLTITLLMTARLWGGTLLWT

SEQ ID NO: 7 - DNA, 2509 bp, Homo sapiens
  ref <308>: DATABASE ACCESSION NUMBER: GenBank / X07393
  ref <309>: DATABASE ENTRY DATE: 1995-03-27
  ref <313>: RELEVANT RESIDUES: (1)..(2509)
gcgaggaaagggaaggagcaagccgtgaatttaagggacgctgtgaagcaatcatggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgtgaccaggaacacccgactcctcaaaagcaaatgagatcccgcctcttcttcttcagaagacactgcaaaggcgcagtgcttctctacagacttctccagacccaccacaccgcagaagcgggacgagaccctacaggagagggaagagtgcattttcccagatacttcccattttggaagttttcaggacttggtctgatttcaggatactctgtcagatgggaagacatgaatgcacactagcctctccaggaatgcctcctccctgggcagaaagtggccatgccaccctgttttcagctaaagcccaacctcctgacctgtcaccgtgagcagctttggaaacaggaccacaaaaatgaaagcatgtctcaatagtaaaagataacaagatctttcaggaaagacggattgcattagaaatagacagtatatttatagtcacaagagcccagcagggcctcaaagttggggcaggctggctggcccgtcatgttcctcaaaagcacccttgacgtcaagtctccttcccctttccccactccctggctctcagaaggtattccttttgtgtacagtgtgtaaagtgtaaatcctttttctttataaactttagagtagcatgagagaattgtatcatttgaacaactaggcttcagcatatttatagcaatccatgttagtttttactttctgttgccacaaccctgttttatactgtacttaataaattcagatatatttttcacagtttttccaaaaaaaaaaa

SEQ ID NO: 8 - protein, 562 aa, Homo sapiens
  ref <308>: DATABASE ACCESSION NUMBER: GenBank / P00750
  ref <309>: DATABASE ENTRY DATE: 1986-07-21
  ref <313>: RELEVANT RESIDUES: (1)..(562)
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 9 - DNA, 1482 bp, Homo sapiens
  ref <308>: DATABASE ACCESSION NUMBER: GenBank / X04429
  ref <309>: DATABASE ENTRY DATE: 1995-03-21
  ref <313>: RELEVANT RESIDUES: (1)..(1482)
ggcagggcaagagcgctgtcaagaagacccacacgcccccctccagcagctgaattcctgcagctcagcagccgccgccagagcaggacgaaccgccaatcgcaaggcacctctgagaacttcaggatgcagatgtctccagccctcacctgcctagtcctgggcctggcccttgtctttggtgaagggtctgctgtgcaccatcccccatcctacgtggcccacctggcctcagacttcggggtgagggtgtttcagcaggtggcgcaggcctccaaggaccgcaacgtggttttctcaccctatggggtggcctcggtgttggccatgctccagctgacaacaggaggagaaacccagcagcagattcaagcagctatgggattcaagattgatgacaagggcatggcccccgccctccggcatctgtacaaggagctcatggggccatggaacaaggacgagatcagcaccacagacgcgatcttcgtccagcgggatctgaagctggtccagggcttcatgccccacttcttcaggctgttccggagcacggtcaagcaagtggacttttcagaggtggagagagccagattcatcatcaatgactgggtgaagacacacacaaaaggtatgatcagcaacttgcttgggaaaggagccgtggaccagctgacacggctggtgctggtgaatgccctctacttcaacggccagtggaagactcccttccccgactccagcacccaccgccgcctcttccacaaatcagacggcagcactgtctctgtgcccatgatggctcagaccaacaagttcaactatactgagttcaccacgcccgatggccattactacgacatcctggaactgccctaccacggggacaccctcagcatgttcattgctgccccttatgaaaaagaggtgcctctctctgccctcaccaacattctgagtgcccagctcatcagccactggaaaggcaacatgaccaggctgccccgcctcctggttctgcccaagttctccctggagactgaagtcgacctcaggaagcccctagagaacctgggaatgaccgacatgttcagacagtttcaggctgacttcacgagtctttcagaccaagagcctctccacgtcgcgcaggcgctgcagaaagtgaagatcgaggtgaacgagagtggcacggtggcctcctcatccacagctgtcatagtctcagcccgcatggcccccgaggagatcatcatggacagacccttcctctttgtggtccggcacaaccccacaggaacagtccttttcatgggccaagtgatggaaccctgaccctggggaaagacgccttcatctgggacaaaactggagatgcatcgggaaagaagaaactccgaagaaaagaattttagtgttaatgactctttctgaaggaagagaagacatttgccttttgttaaaagatggtaaaccagatct

SEQ ID NO: 10 - protein, 402 aa, Homo sapiens [sequence omitted]

SEQ ID NO: 11 - DNA, 31 bp, Artificial Sequence
  PCR primer
atcgaaggccgcccaactctgagtgggattg

SEQ ID NO: 12 - DNA, 28 bp, Artificial Sequence
  PCR primer
tcccaacagcagatctcatgaatgaccc

SEQ ID NO: 13 - DNA, 25 bp, Artificial Sequence
  PCR primer
atgattgaacaagatggattgcacg

SEQ ID NO: 14 - DNA, 24 bp, Artificial Sequence
  PCR primer
ttcgtccagatcatcctgatcgac

SEQ ID NO: 15 - DNA, 20 bp, Artificial Sequence
  PCR primer
tcabcagggcaatgtcacgg

SEQ ID NO: 16 - DNA, 21 bp, Artificial Sequence
  PCR primer
ctctctgtctgccttccatgg

SEQ ID NO: 17 - DNA, 18 bp, Artificial Sequence
  PCR primer
ccgcagggaggtgtgggt

SEQ ID NO: 18 - DNA, 18 bp, Artificial Sequence
  PCR primer
atttcgtggcccagttga

(19) United States
(12) Patent Application Publication
(10) Pub. No.: US 2003/0096733 A1
(43) Pub. Date: May 22, 2003

(54) Novel drug targets for arthritis

(75) Inventors: Tor Ny, Umea (SE); Rikard Holmdahl, Lund (SE); Jinan Li, Umea (SE)

Correspondence Address: DARBY & DARBY P.C., Post Office Box 5257, New York, NY, 10150-5257, US

(73) Assignee: Omnio AB, Umea (SE)

(21) Appl. No.: 10/193,656

(22) Filed: Jul. 10, 2002

Related U.S. Application Data
(60) Provisional application No. 60/304,461, filed on Jul. 10, 2001.
(60) Provisional application No. 60/304,490, filed on Jul. 10, 2001.
(60) Provisional application No. 60/305,182, filed on Jul. 13, 2001.

(51) Int. Cl. A61K 31/00

(52) U.S. Cl. 514/1

(57) ABSTRACT

Novel drug targets for the treatment or prevention of arthritis are provided. Screening methods for inhibitors of the plasminogen-activation pathway, such as, for example, antagonists or inhibitors of the urokinase-type plasminogen activator (uPA), plasminogen-activator type 1 (PAI-1), the urokinase activator receptor (uPAR), and plasmin, are used to identify novel drugs for treating or preventing the progression of arthritis. Such screening methods, or methods for evaluating whether a drug is useful for treating or preventing arthritis, can also be conducted in animal models described herein. Methods of treating or preventing such diseases are also provided.

This application claims the priority of U.S. provisional application serial Nos. 60/304,461, filed Jul. 10, 2001; 60/304,490, filed Jul. 10, 2001; and 60/305,182, filed Jul. 13, 2001. The disclosures of these applications are incorporated herein by reference in their entireties.

### FIELD OF THE INVENTION

This invention relates to arthritic diseases and conditions. In particular, the invention relates to novel treatment or prevention strategies for arthritis such as rheumatoid arthritis. In addition, the invention relates to animal models and screening methods for identifying and evaluating drugs against such drug targets.

### BACKGROUND OF THE INVENTION

Arthritis compromises the quality of life for large numbers of people. For example, more than 5 million people suffer from rheumatoid arthritis (RA) worldwide, of which 2.5 million are in the United States. About 50,000-70,000 children in the United States have been diagnosed with juvenile RA, and psoriatic arthritis affects in the range of 2.5 to 5 million people in the United States alone.

Rheumatoid arthritis (RA) is a systemic chronic autoimmune disease characterized by synovial hyperplasia and inflammatory cell recruitment, intra-articular fibrin deposition, and, in its later stages, cartilage and bone destruction. It is well documented that the degradation of the extracellular matrix (ECM) in bone and cartilage that takes place during the development of RA is dependent on the action of a variety of proteolytic enzymes secreted by both soft and hard tissue cellular elements, as well as by inflammatory cells. Many different proteases are believed to contribute to matrix destruction during RA, although the exact mechanisms responsible for this process and how it is regulated are poorly understood. However, indirect evidence indicates that both matrix metalloproteinases (MMPs) and plasminogen activators (PAs) may play a fundamental role in the pathophysiology of rheumatic disease.

Several MMPs, including interstitial collagenase (MMP-1), stromelysin-1 (MMP-3), and the latent forms of gelatinase A (MMP-2) and gelatinase-B (MMP-9), have been demonstrated in synovial fluid of arthritis patients (Matrisian Trends Genet. 1990;6:121-125; McCachren, Arthritis Rheum 1991;34:1085-93; Koolwijk et al., Arthritis Rheum 1991;34:5143; Hirose et al., J. Rheumatol.; 19:593-599, 1992). MMPs are known to be synthesized as latent precursor enzymes that can be activated by limited proteolysis, but the exact mechanism by which this activation takes place in vivo is largely unknown. It has been suggested, however, that components of the plasminogen-activation system, urokinase-type plasminogen activator and plasmin, might be involved in the activation of specific subclasses of metalloproteinases (Salo et al Int. J. Cancer 1982;30:669-673; Nagase et al., Biochem. Soc. Trans. 1991;19, 715-718).

The plasminogen-activation system is a versatile, temporally controlled enzymatic system in which plasminogen is activated to the proteolytic enzyme plasmin by either of the two physiological plasminogen -activators, tissue-type plasminogen activator (tPA) and urokinase-type plasminogen activator (uPA). uPA is involved in tissue remodeling during wound healing, inflammatory cellular migration, neo-vascularization and tumor cell invasion, while tPA, a key enzyme in thrombosis, is involved in the dissolution of clots in blood vessels and the maintenance of hemostasis in the vasculature. Activation of the plasminogen-activation system is initiated by the release of tPA or uPA by specific cells in response to external signals and leads to a locally expressed extracellular proteolytic activity (Vassalli et al., J. Exp. Med. 1977;146:857-868; Saksela & Rifkin, Annu. Rev. Cell Biol. 1988;4:93-126). The PA-system is also regulated by specific inhibitors directed against PAs and plasmin, including PA-inhibitor type 1 (PAI-1), PA-inhibitor type 2 (PAI-2), protease nexin 1 (PN-1) and a 2-anti-plasmin (Saksela & Rifkin, Annu. Rev. Cell Biol. 1988;4:93-126; Ny et al., Thromb. Res. 1993;71:1-45). All of these inhibitors, which belong to the serpin family, are suicide inhibitors that are cleaved by cognate protease (Wilczynska et al., Nature Struct. Biol. 1997;4:354-357). The most important feature of the PA-plasmin system is the amplification achieved by the conversion of plasminogen to plasmin. Because of the high concentration of plasminogen in virtually all tissues, the production of relatively small amounts of PA can result in high local concentrations of plasmin.

Both MMPs and plasminogen activators (PAs) are present in affected joints and their expression is induced by inflammatory mediators and cytokines, indicating that the two enzyme systems may act in concert (for reviews see Hart and Fritzler J Rheumatol,; 16:1184-1191, 1989 and Matthews Arthritis and Rheum. 1994;37:1115-1126). Furthermore, a number of reports have indicated that the expression of MMPs, tissue inhibitors of metalloproteinases (TIMPs), PAs and PA-inhibitors are altered in rheumatic diseases, which often lead to increased activity of proteases capable of degrading cartilage proteoglycans as well as other cartilage and bone matrix proteins. Data suggesting that arthritis is exacerbated by lack of uPA, however, based upon an in vivo model, have also been presented (Busso et al., 1998). In some cases it also seems like there is an imbalance in the expression of MMPs versus TIMP and PAs versus PA-inhibitors (Hart and Fritzler, J Rheumatol; 16:1184-1191, 1989, Koolwijk et al., Arthritis Rheum 1991;34:5143, Ahrens et al., Arthritis and Rheum. 39(9):1576-1587 (1996).

Accumulation of intraarticular fibrin, resulting from the altered balance between coagulation and fibrinolysis, is a common feature of RA and it is possible that these fibrin deposits can have adverse effects (Weinberg et al., Ann. Rheum. Dis. 1991;56:550-557; Jasini, In: Immunopathogenesis of Rheumatoid Arthritis. G. S. Panayi and P. M. Johnson (Eds.), Red Books, Surrey. 1991,137-146). In this context, degradation of fibrin matrix, which is mainly performed by plasmin, could be beneficial. The possibility that plasmin may, in fact, play a beneficial role in intra-articular fibrin removal has only recently been discussed (Busso et al., J. Clin. Invest; 102: 41-50, 1998).

Thus, there is a need in the art for new and improved methods for treating or preventing arthritis and other conditions leading to tissue destruction and bone loss. There is also a need in the art for new screening methods that can be employed to identify and evaluate drugs for use in such treatment methods. The invention addresses these and other needs in the art.

### SUMMARY OF THE INVENTION

The present invention provides novel drug targets which can be used for new and improved treatment or prevention strategies for arthritis, and for methods of screening for, or evaluating the usefulness of, drugs directed against such drug targets.

One aspect of the present invention involves methods for screening test compounds to identify agents that inhibit plasmin formation or activity, plasminogen formation or activation into plasmin, urokinase-type plasminogen activator (uPA) formation or activity, uPA receptor (uPAR) formation or ligand binding, or plasminogen-activator inhibitor type 1 (PAI-1) formation or activity. Another aspect of the invention involves active pharmaceutical agents that inhibit one or more drug targets such as plasmin, plasminogen, uPA, uPAR, and PAI-1. Yet another aspect involves pharmaceutical agents, active in treating or preventing arthritis.

Accordingly, the invention provides a method of treating or preventing arthritis in a mammal, which comprises administering to the mammal an effective amount of an agent that inhibits at least one member selected from the group consisting of plasmin, plasminogen, urokinase-type plasminogen activator, urokinase-type plasminogen activator receptor, and plasminogen-activator inhibitor type 1. In one embodiment, the agent comprises a protease inhibitor, such as, for example, aprotinin. In another embodiment, the agent comprises amiloride. In still other embodiments, the agent is a monoclonal antibody directed against the at least one member, or an anti-sense nucleic acid sequence capable of binding to a nucleic acid encoding the at least one member. The arthritis can be caused by a degenerative joint disease, such as for example, rheumatoid arthritis, psoriatic arthritis, infectious arthritis, juvenile rheumatoid arthritis; osteoarthritis, or spondyloarthropaties. In a preferred embodiment, the arthritis is rheumatoid arthritis, and the mammal is a human.

The invention also provides a method of screening to identify an agent useful for treating or preventing arthritis, which comprises (i)providing a pool of test agents; (ii) determining whether any test agent from the pool inhibits the activity of at least one member selected from the group consisting of plasmin, plasminogen, urokinase-type plasminogen activator, urokinase-type plasminogen activator receptor, and plasminogen-activator inhibitor type 1, and (iii) selecting any test agent from the pool that inhibits the activity of at least one member as an agent useful for treating or preventing arthritis. The method may optionally comprise a step of selecting the pool of test agents prior to step (i).

In a first embodiment, the determining step comprises (i) contacting a test agent from the pool with plasmin and a substrate to form a product; (ii) measuring the level of the substrate or the product after the contacting step; (iii) comparing the substrate level to a substrate control value or the product level to a product control value; and (iv) selecting any test agent for which the substrate level is higher than the substrate control value or for which the product level is lower than the product control value as an agent useful in treating or preventing arthritis. Optionally, the substrate is, for example, H-D-Valyl-L-leucyl-L-lysine-p-nitroaniline, and the product p-nitroaniline dihydrochloride, and the plasmin may be formed by contacting plasminogen with a plasminogen activator.

In a second embodiment, the determining step comprises (a) contacting a test agent from the pool with urokinase-type plasminogen activator and a substrate to form a product; (b) measuring the level of the substrate or the product after the contacting step; (c) comparing the substrate level to a substrate control value or the product level to a product control value; and (d) selecting any test agent for which the substrate level is higher than the substrate control value or for which the product level is lower than the product control value as an agent that inhibits the urokinase-type plasminogen activator. Optionally, the substrate is L-pyroglutamyl-glycyl-L-arginine-p-nitroaniline hydrochloride and the product p-nitroaniline dihydrochloride.

In a third embodiment, the determining step comprises (a) contacting the test agent with urokinase-type plasminogen activator receptor and urokinase-type plasminogen activator; (b) measuring the level of binding between the urokinase-type plasminogen activator receptor and urokinase-type plasminogen activator substrate after the contacting step; (c) comparing the level of binding to a control value; and (c) selecting any test agent for which the level of binding is lower than the control value as an agent useful for treating or preventing arthritis.

In a fourth embodiment, the determining step comprises (a) contacting the test agent with plasminogen-activator inhibitor type 1, an excess amount of urokinase-type plasminogen activator, and a substrate to form a product; (b) measuring the level of the substrate or the product after the contacting step; (c) comparing the substrate level to a substrate control value or the product level to a product control value; and (d) selecting any test agent from the pool for which the substrate level is lower than the substrate control value or the product level is higher than the product control value as an agent capable of inhibiting the plasminogen-activator inhibitor type 1. Optionally, the substrate is L-pyroglutamyl-glycyl-L-arginine-p-nitroaniline hydrochloride and the product p-nitroaniline dihydrochloride.

In a fifth embodiment, the test agent comprises an antigen-binding fragment of an antibody directed against the at least one protein. Also, the method may optionally comprise selecting any test agent that inhibits the formation of plasmin from plasminogen as an agent useful in treating or preventing arthritis. Alternatively, the test agent inhibits the ability of urokinase-type plasminogen activator to promote formation of plasmin from plasminogen.

The invention also provides for a method of identifying an agent that is useful in preventing or treating arthritis, which comprises (i) administering a test agent to a transgenic animal susceptible to collagen-induced arthritis, said animal lacking endogenous expression of at least one protein selected from the group consisting of plasmin, plasminogen, urokinase-type plasminogen activator, urokinase-type plasminogen activator receptor, and plasminogen-activator inhibitor type 1; (ii) administering a human homolog of the at least one protein to the animal; (iii) administering type II collagen to the animal to induce collagen induced arthritis in the animal; (iv) determining the severity level of the induced collagen-induced arthritis in the animal; (v) comparing the severity level to a control value; and (vi) selecting any test agent for which the severity level is lower than the control value as an agent that is useful in preventing or treating arthritis. Optionally, the control value is the severity level of collagen-induced arthritis in a control animal.

The invention also provides for a method of identifying an agent as useful in treating rheumatoid arthritis which comprises administering a test agent to a mammal and determining whether the test agent inhibits plasmin, plasminogen, urokinase-type plasminogen activator, urokinase-type plasminogen activator receptor, or plasminogen-activator inhibitor type 1 in said mammal.

The invention also provides for the use of a composition comprising an effective amount of an agent that inhibits at least one member selected from the group consisting of plasmin, plasminogen, urokinase-type plasminogen activator, urokinase-type plasminogen activator receptor, and plasminogen-activator inhibitor type 1; and a pharmaceutically acceptable carrier, in the manufacture of a medicament for administration to a mammal to treat or prevent arthritis. The mammal may optionally be a human.

The above features and many other advantages of the invention will become better understood by reference to the following detailed description when taken in conjunction with the accompanying drawings.

### BRIEF DESCRIPTION OF THE DRAWINGS

FIG. 1. This figure shows a comparison between the daily arthritis scores for the uPA-deficient and uPA wild type mice after induction of collagen-induced arthritis.

FIG. 2. This figure depicts the incidence of CIA in wild-type and uPA deficient mice.

FIG. 3. This figure depicts the daily arthritis scores for plasminogen deficient mice as compared to wild-type mice.

FIG. 4. This figure depicts the incidence of arthritis in wild-type, plasminogen heterozygous and homozygous mice.

FIG. 5. This figure depicts the incidence of collagen-induced arthritis in plasminogen activator inhibitor type 1 (PAI-1) heterozygous or knock-out mice as compared to wild-type mice

FIG. 6. This figure depicts the severity of collagen-induced arthritis in PAI-1 heterozygous or knock-out mice as compared to wild-type mice.

FIG. 7. This figure depicts the severity of arthritis in tPA wild-type and tPA deficient mice. The chart shows that there was no difference in the severity or incidence of arthritis between tPA deficient and wild-type control mice.

FIG. 8. This figure depicts the incidence of arthritis in tPA wild-type and tPA deficient mice. The chart shows that there was no difference in the severity or incidence of arthritis between tPA deficient and wild-type control mice.

FIG. 9. This figure depicts the severity of arthritis in uPAR wild-type and uPAR deficient mice. The chart shows that the severity of arthritis was lower in the uPAR deficient mice.

FIG. 10. This figure depicts the incidence of arthritis in uPAR wild-type and uPAR deficient mice. The chart shows that the incidence of arthritis was lower in the uPAR deficient mice.

### DETAILED DESCRIPTION OF THE INVENTION

According to the present invention, inhibiting the formation or activity of drug targets such as plasmin, plasminogen, urokinase-type plasminogen activator (uPA), the uPA receptor (uPAR), or PAI-1 can prevent or reduce the development or progression of arthritis. This provides for new treatment and prevention strategies for arthritis, as well as new screening and evaluation methods for drugs to be used in such treatment or prevention methods.

The invention is, at least in part, based on the findings described in the Examples. As described in Examples 1-4, collagen type II-induced arthritis (CIA) was reduced in mice lacking either uPA, uPAR, plasminogen, or PAI-1, thus showing that these components play pivotal roles in the development of CIA. More specifically, in studies utilizing the CIA model in CIA sensitive DBA/1 mice, it was discovered that the development of CIA was attenuated in mice lacking uPA as compared to wild-type controls (see Example 1). Moreover, those uPA deficient mice which showed symptoms of arthritis only developed less severe forms of the disease, and mice lacking plasminogen did not develop CIA at all during a 60 day test period. It was also found that plasminogen-deficient mice that were resistant to the development of RA became prone to the disease following injection of human plasminogen, and PAI-1 was also shown to play a role in the pathology of arthritis (see Example 2).

To investigate if the immune response towards collagen type II was affected in plasminogen deficient mice, collagen II antibody levels were determined in wild-type and plasminogen deficient mice 60 days after the boost injection. Although none of the plasminogen deficient mice developed arthritis there was no significant difference in antibody levels between wild-type and plasminogen deficient mice. Immunostaining experiments further showed different subpopulations of inflammatory cells in tissue sections from mice with different genotypes. Therefore, without being limited to any specific mechanism, it is believed that since the plasminogen deficient mice do not develop any inflammation, although they have collagen II antibodies, the inflammatory response or the recruitment of inflammatory cells might be defective due to the lack of plasminogen. In the case of PAI-1, which also seems to be required for the development of arthritis, this molecule is known to play a role in cell adhesion and migration in addition to its role as an inhibitor of PAs. It is therefore possible that the reason why lack of uPA, uPAR, plasminogen, plasmin, or PAI-1 reduces induction of arthritis is that inflammatory cells cannot invade joints.

### Definitions

The terms used in this specification generally have their ordinary meanings in the art, within the context of this invention and in the specific context where each term is used. Certain terms are discussed below, or elsewhere in the specification, to provide additional guidance to the practitioner in describing the methods of the invention and how to use them.

“Arthritis” as used herein means all conditions characterized by inflammation of one or more joints. Any disease or disorder associated with joint inflammation, tissue destruction, and/or degeneration of extracellular matrix structures, particularly joint cartilage and bone, may cause arthritis. Such conditions include, without limitation, rheumatoid arthritis (RA); psoriatic arthritis, infectious arthritis, juvenile rheumatoid arthritis; osteoarthritis, and spondyloarthropaties. Symptoms of arthritis include, but are not limited to, swelling, warmth, redness of the overlying skin, pain, and restriction of motion. Arthritis can be monitored or diagnosed by X-ray or blood analysis, examination of synovial fluid taken from affected joints, and, according to the American Rheumatism Association criteria for classification of arthritis, diagnosed as follows: A patient is said to have arthritis if he or she has satisfied at least 4 of the following 7 criteria. Criteria 1 through 4 must have been present for at least 6 weeks. Patients with 2 clinical diagnoses are not excluded. (1) Morning stiffness—Morning stiffness in and around the joints, lasting at least 1 hour before maximal improvement; (2) Arthritis of 3 or more joint areas—At least 3 joint areas simultaneously have had soft tissue swelling or fluid (not bony overgrowth alone) observed by a physician; the 14 possible joint areas are right or left proximal interphalangeal (PIP) joints, metacarpophalangeal (MCP) joints, wrist, elbow, knee, ankle, and metatarsophalangeal (MPT) joints; (3). Arthritis of hand joints—At least 1 area swollen (as defined above) in a wrist, MCP or PIP joint; (4) Symmetric arthritis—Simultaneous involvement of the same joint areas (see No. 2 above) on both sides of the body (bilateral involvement of PIPs, MCPs, or MTPs is acceptable without absolute symmetry); (5) Rheumatoid nodules—Subcutaneous nodules, over bony prominences, or extensor surfaces, or in juxta-articular regions, observed by a physician; (6) Serum rheumatoid factor—Demonstration of abnormal amounts of serum rheumatoid factor by any method for which the result has been positive in <5% of normal control subjects; and (7) Radiographic changes—Radiographic changes typical of RA on posteroanterior hand and wrist radiographs, which must include erosions or unequivocal bony decalcification localized to or most marked adjacent to the involved joints (osteoarthritis changes alone do not qualify).

Successful “treatment” of arthritis means that the extent of arthritis (evaluated by, for example, X-ray diagnosis, sampling of synovial fluid, or ease of movement of the joint) in a particular joint is less after the treatment than before. Successful treatment of arthritis can also be that a patient satisfies less criteria after a treatment than before, according to the criteria listed above. Alternatively, successful treatment of arthritis can be that a patient which before treatment satisfied 4 or more of the criteria above, satisfies less than 4 criteria after the treatment.

The term “extracellular matrix” (ECM) means the noncellular portion of animal tissues. The ECM of connective tissue is particularly extensive and the properties of the ECM determine the properties of the tissue. In broad terms there are three major components: fibrous elements particularly collagen, elastin or reticulin), link proteins (e.g., fibronectin, laminin) and space filling molecules (usually glycosaminoglycans). The matrix may be mineralized to resist compression (as in bone) or dominated by tension resisting fibers (as in tendon).

The term “inhibitor” refers to a molecule that directly or indirectly decreases the biological activity or level (i.e., amount or concentration in blood or in joints, particularly arthritic joints) of a target protein. The inhibitor may be any type of compound, including, but not limited to, an organic or inorganic molecule, a peptide, a protein, an anti-sense nucleic acid, and a polyclonal or monoclonal antibody preparation. An “indirect” inhibitor is a molecule that does not bind to the target protein, but decreases its biological activity or level in an indirect manner, e.g., by reducing transcription of the gene encoding the target protein, or binds to the transcribed MRNA thus preventing translation into the target protein. A “direct” inhibitor is a compound which binds to the target protein, thus directly inhibiting the activity of the target protein.

The “activity” of a protein means the ability of a protein to participate in a biochemical pathway in vivo. For example, the proteins plasmin, plasminogen, uPA, uPAR, and PAI-1 all participate in the plasminogen activation pathway. For proteins having enzymatic activity, i.e., capability to promote the conversion of a substrate into a product, the “activity” means the enzymatic activity. uPA and plasmin both have enzymatic activity. For example, for tPA and uPA, “activity” can mean the capability to convert plasminogen to plasmin, whereas for plasmin, “activity” can mean the capability to degrade a substrate such as fibrin or chromogenic substrates. For a receptor such as uPAR, “activity” means the capability to bind an agonist receptor ligand such as uPA. For plasminogen, “activity” means the capability to be converted into plasmin in the presence of a plasminogen activator such as uPA or tPA. For PAI-1, “activity” means the capability to inhibit uPA activity.

“Transgenic animal” is any animal, preferably a non-human mammal in which one or more of the cells of the animal contain heterologous nucleic acid, “transgene”, introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of the coded protein.

“Mammals” include both humans and non-human mammals. Non-human mammals include, without limitation, laboratory animals such as mice, rats, rabbits, hamsters, guinea pigs, etc.; domestic animals such as dogs and cats; and, farm animals such as sheep, goats, pigs, horses, and cows.

The term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

Abbreviations

Abbreviations used in the present disclosure include the following:

CIA=Collagen type II-induced arthritis;

uPA=Urokinase-type plasminogen activator;

uPAR=Urokinase-type plasminogen activator;

PA=Plasminogen activator;

RA=Rheumatoid arthritis;

MMP=Matrix metalloproteinase;

TIMP=Tissue inhibitor of metalloproteinase;

tPA=Tissue-type plasminogen activator;

FACS=Fluorescence-activated cell sorting;

CII=Collagen type II.

Plg=Plasminogen

PAI-1=Plasminogen activator inhibitor type-1

### Plasminoten Activation System

The plasminogen activation pathway is the pathway leading to the formation of active plasmin in mammals. The plasminogen-activation pathway includes, but is not limited to, the following components: plasminogen, plasmin, tPA, uPA, PAI-1, protease nexin 1 (PN-1) and (α₂-anti-plasmin.

Briefly, plasminogen is activated by uPA or tPA-catalyzed cleavage between Arg-560 and Val-561, to form plasmin. After the cleavage, plasmin is an active two-chain disulfide linked molecule. There are two forms of both plasminogen and plasmin. The full-length forms are called glu-plasminogen or glu-plasmin and the shorter forms are cleaved between residues Lys-76 and Lys-77. The shorter forms are called lys-plasminogen and lys-plasmin respectively. PAI-1, PAI-2, and protease-nexin 1 regulate the activity of the two plasminogen activators.

Alpha2-anti-plasmin is a single-chain glycoprotein with a molecular mass of 67 kD that is a physiological inhibitor of plasmin. This protein is synthesized by the liver and the concentration in plasma is approximately 70 μg/ml, which is about half to one third of the concentration of plasminogen in plasma. The inhibition of plasmin by α2AP is very fast, with a second-order rate constant above 10⁷M⁻¹s⁻¹. α2AP is a single-chain glycoprotein composed of 452 amino acid residues, with a molecular mass of approximately 60 kda. The structure of a2AP has several unique features, which make it a unique molecule. For example, the amino terminal region contains 4 disulfide bonded systeine residues forming a special secondary structure, and the carboxy terminal part has an extension of about 50 amino acid residues. During coagulation, α2AP can be cross-linked by factor XIII to fibrin. This reaction may be important for prevention of premature lysis of blood clots. Cross-linking occurs between glutamic acid in the amino terminal part of α2AP and lysine in the α-chain of fibrin. In circulation, this mechanism is thought to ensure that plasmin activity is restricted to fibrin.

The following table (Table 1) provides exemplary, non-limiting, nucleotide and protein sequences, identified by GenBank Accession numbers, for various components of this pathway in humans. Naturally occurring variants or mutants of these human sequences are known in the art.

TABLE 1
Nucleic acid and amino acid sequences for components
of the plasminogen activation pathway

| Component | Nucleic Acid Sequence | Amino Acid Sequence |
|---|---|---|
| Plasminogen | X05199 (SEQ ID NO:1) | P00747 (SEQ ID NO:2) |
| uPA | X02419 (SEQ ID NO:3) | P00749 (SEQ ID NO:4) |
| uPAR | X51675 (SEQ ID NO:5) | Q03405 (SEQ ID NO:6) |
| tPA | X07393 (SEQ ID NO:7) | P00750 (SEQ ID NO:8) |
| PAI-1 | X04429 (SEQ ID NO:9) | P05121 (SEQ ID NO:10) |

### In Vitro Screening Methods

Various assays can be designed to screen for inhibitors of uPA, uPAR, Plg, plasmin and/or PAI-1. Although in vitro methods are preferred for any initial screening of large number of potential drug candidates or agents, the in vivo methods described below may also be used for screening.

### Screening for Indirect Inhibitors

The inhibitors may be both direct and indirect inhibitors. Preferred, although non-limiting, examples of indirect inhibitors include anti-sense nucleic acids complementary to genomic DNA or MRNA encoding uPA, uPAR, plasminogen or PAI-1, thus preventing translation of the coding nucleic acid sequences into the target protein. Methods to design and screen for antisense nucleic acids are well-known in the art. Thus, anti-sense sequences may be used to modulate the activity of the drug target or to achieve regulation of gene function. Sense or anti-sense oligomers, or larger fragments, can be designed from various locations along the coding or regulatory regions of sequences encoding a drug target of the invention.

Alternative indirect inhibitors include compounds that reduce transcription of the genes encoding the target protein. Gene expression may be down-regulated by treating the patient with drugs, hormones, cytokines, etc. Both the uPA and PAI-1 genes are regulated by many different agents known in the art.

### Screening for Direct Inhibitors

Inhibition of the target proteins uPA, uPAR, Plg, and PAI-1, can be determined by evaluating the inhibitory effect of a drug candidate or test agent on the biological activity of the selected target protein (“drug target”) in comparison to a control or reference. The control or reference may be a predetermined reference value, or may be evaluated experimentally. For example, the control or reference value can be a measure of the biological activity of the target protein in the absence of the test agent, or the biological activity of a reference protein in the presence of test agent, or any other suitable control or reference.

Drugs or agents that inhibit the activity of a target protein can be identified based on their ability to associate with the drug target protein. Association with a drug target can be tested by reacting a drug target protein or fragment with a test substance which has the potential to associate with the drug target under appropriate conditions, and removing and/or detecting the associated drug target/test substance complex. Binding may be detected by indirect or direct functional measures such as alteration of migration pattern in protein gel electrophoresis, immunoprecipitation, or the Biomolecular Interaction Assay (BIAcore; Pharmacia). A drug candidate that associates with a drug target protein of the invention is preferably an antagonist or inhibitor of the biological activity of a drug target, as shown by an activity assay.

Activity assays are generally designed to measure the activity of a target protein in the presence or absence of a test agent. Many different activity assays may be designed based on various art-recognized methods for studying the activity of plasmin, plasminogen, uPA, uPAR, and PAI-1. For example, as described in Examples 5 and 6, inhibitors of uPA or plasmin activity can be identified by measuring the ability of uPA or plasmin to promote the conversion of a substrate into a chromogenic, fluorogenic, or otherwise detectable product, over a suitable period of time. Optionally, in cases where the substrate is detectable by absorbance, fluorescence, or by coloring, the amount of intact substrate remaining can be measured after incubation with uPA or plasmin for suitable time period.

Inhibitors of plasminogen activation activity, i.e., the ability of plasminogen to be converted into plasmin, can be studied in an assay in which plasminogen is mixed with a plasminogen activator such as tPA or uPA, and formation of plasmin indicated by use of a chromogenic assay (see Example 6). uPAR activity can be studied by measuring the ability of uPAR to bind uPA, using any of the binding evaluation methods described above, the assay outlined in Example 7, or any other suitable method of measuring uPA binding to uPAR that is known in the art. PAI-1 activity can advantageously be evaluated in an assay similar to the assay described for uPA above, but including PAI-1 in the assay system (Example 8). Thus, in the absence of a PAI-1 inhibitor, PAI-1 inhibits uPA conversion of a substrate into a detectable product. Conversely, in the presence of a PAI-1 inhibitor, PAI-1 is no longer capable of inhibiting uPA function, and detectable product is thereby formed.

An exemplary method of identifying an agent that is useful in preventing or treating arthritis, particularly a method that detects inhibition of plasmin, comprises (i) providing a pool of test agents; (ii) mixing a test agent from the pool with plasmin and H-D-Valyl-L-leucyl-L-lysine-p-nitroaniline dihydrochloride, under conditions suitable for forming p-nitroaniline dihydrochloride from the H-D-Valyl-L-leucyl-L-lysine-p-nitroaniline dihydrochloride; (iii) incubating the mixture for a predetermined time period; (iv) measuring a test absorbance of the mixture at 405 nm; (v) comparing the test absorbance with a control absorbance; and (vi) selecting any test agent for which the test absorbance is lower than the control absorbance as an agent that is useful in treating or preventing arthritis. The predetermined time period can be 4 hours, and the mixture is incubated at about 37° C. Optionally, the control absorbance is the absorbance of a mixture of plasmin and H-D-Valyl-L-leucyl-L-lysine-p-nitroaniline dihydrochloride.

Another exemplary assay, particularly for screening for an agent that inhibits the activation or activity of plasminogen, comprises (i) providing a pool of test agents; (ii) mixing a test agent from the pool with plasminogen, an excess amount of urokinase-type plasminogen activator, and H-D-Valyl-L-leucyl-L-lysine-p-nitroaniline dihydrochloride, under conditions suitable for forming p-nitroaniline dihydrochloride from the H-D-Valyl-L-leucyl-L-lysine-p-nitroaniline dihydrochloride; (iii) incubating the mixture for a predetermined time period; (iv) measuring a test absorbance of the mixture at 405 nm; (v) comparing the test absorbance with a control absorbance; and (vi) selecting any test agent for which the test absorbance is lower than the control absorbance as an agent useful in treating or preventing arthritis. Optionally, the predetermined time period is about 4 hours, and the mixture is incubated at about 37° C. The control absorbance can be the absorbance of a mixture of plasminogen, an excess amount of urokinase-type plasminogen activator, and H-D-Valyl-L-leucyl-L-lysine-p-nitroaniline dihydrochloride.

A third exemplary assay, for identifying an agent that is useful in preventing or treating arthritis, which comprises: (i) providing a pool of test agents; (ii) mixing a test agent from the pool with urokinase-type plasminogen activator and L-pyroglutamyl-glycyl-L-arginine-p-nitroaniline hydrochloride, under conditions suitable for forming p-nitroaniline dihydrochloride from the L-pyroglutamyl-glycyl-L-arginine-p-nitroaniline hydrochloride; (iii) incubating the mixture for a predetermined time period; (iv) measuring a test absorbance of the mixture at 405 nm; (v) comparing the test absorbance with a control absorbance; and (vi) selecting any test agent for which the test absorbance is lower than the control absorbance as an agent useful in treating or preventing arthritis. Optionally, the predetermined time period is about 0.5 hours, and the mixture is incubated at about 37° C. Also, the control absorbance can be the absorbance of urokinase-type plasminogen activator and L-pyroglutamyl-glycyl-L-arginine-p-nitroaniline hydrochloride.

Another exemplary assay, particularly for identifying an inhibitor of uPAR that is useful in preventing or treating arthritis, comprises (i) contacting a test agent with human urokinase-type plasminogen activator and an murine cell expressing a human urokinase-type plasminogen activator receptor, under conditions suitable for association of the human urokinase-type plasminogen activator to the receptor; (ii) contacting the murine cell with a casein plaque; and (iii) selecting any test agent for which the casein plaque is not degraded by the contacting in step (ii) as an agent useful in treating or preventing arthritis. Optionally, the method comprises selecting the test agent from a plurality of test agents.

Yet another exemplary screening method, particularly for identifying a PAI-1 inhibitor that is useful in preventing or treating arthritis, comprises (i) providing a pool of test agents; (ii) mixing a test agent with plasminogen-activator inhibitor type 1, urokinase-type plasminogen activator and L-pyroglutamyl-glycyl-L-arginine-p-nitroaniline hydrochloride, under conditions suitable for forming p-nitroaniline dihydrochloride from the L-pyroglutamyl-glycyl-L-arginine-p-nitroaniline hydrochloride; (iii) incubating the mixture for a predetermined time period; (iv) measuring a test absorbance of the mixture at 405 mn; (v) comparing the test absorbance with a control absorbance; and (vi) selecting an agent useful in treating or preventing arthritis any test agent for which the test absorbance is higher than the control absorbance. Optionally, the predetermined time period is about 0.5 hours, and the mixture is incubated at about 23° C., and the control absorbance can be the absorbance of plasminogen-activator inhibitor type 1, urokinase-type plasminogen activator and L-pyroglutamyl-glycyl-L-arginine-p-nitroaniline hydrochloride, incubated for 0.5 hours at 23° C.

Many different variations of the methods described above and in the Examples, using different time periods, chromogenic substrates or products, or detection methods, will be apparent to those skilled in the art.

### Hiah-Throughput Screening

The in vitro assay systems described here may be used in a high-throughput primary screen for compounds. For example, drug candidates according to the invention may advantageously be identified by screening in high-throughput assays, including without limitation cell-based or cell-free assays. It will be appreciated by those skilled in the art that different types of assays can be used to detect different types of drugs or agents. Several methods of automated assays have been developed in recent years so as to permit screening of tens of thousands of compounds in a short period of time (see, e.g., U.S. Pat. Nos. 6,303,322, 5,585,277, 5,679,582, and 6,020,141). Such high-throughput screening methods are particularly preferred. Identifying agents is greatly facilitated by use of high-throughput screening assays to test for agents together with large amounts of drug candidates, provided as described herein.

### In Vivo Screening Methods

In one embodiment of the invention, drugs that inhibit the activity or formation of uPA, uPAR, Plg, plasmin and/or PAI-1are identified, tested, or optimized for preventing formation of arthritis in normal collagen type-II induced arthritis (CIA) -sensitive wild-type or transgenic mice. The CIA or transgenic animal assay system are utilized to test for agents or drugs that reduce or inhibit arthritis by inhibiting or reducing the expression or activity of drug target proteins such as plasmin, plasminogen, uPA, uPAR, and PAI-1.

CIA is today the most commonly used model for RA (Trentham et al., J. Exp. Med. 1977;146:857-868; Holmdahl et al., Lab Invest.; 58, 53-60, 1988) and is widely accepted in the field. For example, the DBA/1 mouse strain is genetically susceptible to RA, which can be induced by homologous and heterologous type II collagen or antibodies to type II collagen (See Example 1). The resulting condition is an erosive inflammatory disease affecting peripheral joints, and tissue distribution and histopathology of the destruction process mimics that of RA. The susceptibility of this model is associated with MHC class II genes. Accordingly, transgenic animals based on the CIA model can be prepared for evaluating potential drugs affecting the onset or progression of RA. Such animals provide excellent models for screening or testing drug candidates. In CIA animal models, the severity of the CIA can be tracked using a scoring system which defines one inflamed toe or knuckle as 1 point and one inflamed wrist or ankle as 5 points, resulting in a score of 0-15 points for each paw and 0-60 points per mouse. Deformed or swollen without redness are generally not included in this system. The severity of arthritis can be evaluated at a suitable time point, usually after at least 1, preferably at least 10, and most preferably at least 20 days, after the injection of a boosting agent that enhances the antibody response to CII. The boosting agent can be administered a few days, e.g., about 2 days or 5 days, after administration of CII or anti-CII antibodies, respectively.

The in vivo models of the invention can advantageously be used for testing the efficacy of a drug identified as a candidate drug in an in vitro screen, optimizing dosages and administration schedules of the drug candidate to inhibit the development or progression of CIA-induced arthritis. The screening method of the invention also encompasses determining whether a test drug shows an inhibitory effect with regard to the binding of uPA to its cellular receptor, the urokinase receptor (UPAR), an antagonist effect being indicative of a drug useful for preventing or treating degradation of extracellular matrix.

### Wild-Type CIA Model

When using wild-type CIA mice, the mice may be treated with a selected test drug, or a drug candidate identified in a previous screen, and the incidence or severity of arthritis upon injection of collagen may be monitored and compared with control animals. If the mice develop arthritis, the tested compound interferes with or targets some component that is important for the development of arthritis. Alternatively, the drug may be administered after the induction of arthritis, to study whether the drug can reduce the symptoms associated with CIA. An example of such an assay is provided in Example 9.

### Transeenk Animal Model

Transgenic animals for use in the present invention can be prepared by any method, including, but not limited to, modification of embryonic stem (ES) cells and heteronuclear injection into blast cells, and such methods are known in the art (see, e.g., Cofftnan, Semin. Nephrol. 17:404, 1997; Esther et al., Lab. Invest. 74:953, 1996; Heddle, Environ Mol Mutagen 32:110-4, 1998; Werner et al., Arzneimittelforschung 48:870-80, 1998; U.S. Pat. Nos. 4,736,866 (Leder and Steward); 4,870,009 (Evans et al.); 5,718,883 (Harlan and June); 5,614,396 (Bradley et al.); and 5,650,503 (Archibald et al.). Preferably, the transgenic animal model is based on collagen induced arthritis (CIA)-sensitive mice, and collagen is administered before, in conjunction with, or after the administration of the test drug.

A “knockout mammal” is a mammal (e.g., mouse) that contains within its genome a specific gene that has been inactivated by the method of gene targeting (see, e.g., U.S. Pat. No's. 5,777,195 and 5,616,491). A knockout mammal may be either a heterozygote knockout (i.e., one defective allele and one wild-type allele) or a homozygous mutant. Plasminogen, PAI-1, and uPA-deficient (“knock-out”) mice can be prepared according to Ploplis et al (Circulation 1995, 92:2585-2593), Carmeliet et al. (J Clin Invest. 1993, 92:2746-60), and Carmeliet et al. (Nature 1994, 368:419-424), respectively. In the method of the invention, a “knock-out” animal is preferably given a heterologous, preferably human, counterpart is instead by administering the target protein systemically. For example, human plasminogen could be administered locally or systemically in a plg−/−animal before, during, or after administration of a drug candidate. After administration of a test drug and CII, the severity and/or RA-incidence be determined. Optionally, the activity of uPA, uPAR, plasminogen, plasmin, and/or PAI-1 can be measured.

A “knock-in” mammal is a mammal in which an endogenous gene is substituted with a heterologous gene (Roamer et al., New Biol. 1991;3:331). Preferably, the heterologous gene is “knocked-in” to a locus of interest, either the subject of evaluation (in which case the gene may be a reporter gene; see Elegant et al., Proc. Natl. Acad. Sci. USA; 95:11897, 1998) of expression or function of a homologous gene, thereby linking the heterologous gene expression to transcription from the appropriate promoter. This can be achieved by homologous recombination, transposon (Westphal and Leder, Curr Biol 1997;7:530), using mutant recombination sites (Araki et al., Nucleic Acids Res, 25:868; 1997) or PCR (Zhang and Henderson, Biotechniques 1998;25:784).

For example, transgenic “knock-in” animals can be created in which (i) a human uPA, uPAR, plasminogen, and/or PAI-1 gene is stably inserted into the genome of the transgenic animal; and/or (ii) the endogenous corresponding genes are inactivated and replaced with their human counterparts (see, e.g., Coffman, 1997; Esther et al., 1996; and Murakami et al., 1996). Preferably, the animals are susceptible or inducible to develop arthritis or another ECM destructive disease, such as, for example, CIA mice (see, e.g., Wang et al., J Immunol 2000; 164:4340-4347). Such animals can then be treated with candidate compounds and monitored for RA development or level/activity of selected proteins or enzymes, for example by (a) administering the agent to a wild-type or transgenic non-human animal of the invention; (b) inducing arthritis, and (c) determining whether said agent reduces or inhibits the arthritis pathology in the wild-type or transgenic non-human animal relative to a wild-type or transgenic non-human animal of step (a) to which the agent has not been administered. Advantageously, this type of model can be used for testing efficacy and suitable dosages of anti-sense nucleotides directed against the gene or mRNA encoding the human target protein.

The agents identified as reducing the onset or progression of arthritis in these models can be used to treat the disorders and conditions discussed herein. The agents may also be incorporated in a pharmaceutical composition as described herein.

### Drug Candidates

Any type of compound or compound library can be screened for efficacy in inhibiting uPA, uPAR, PAI-1, and/or plasminogen formation or activity according to the invention, to identify drugs that are useful in preventing or treating arthritis or other tissue-degenerative diseases.

For example, the present invention contemplates methods for screening for small molecules and mimics, as well as methods for screening for natural products that inhibit uPA, uPAR, PAI-1, plasmin and/or plasminogen formation or activation. Natural products libraries can be screened using assays of the invention for molecules that inhibit the drug targets identified herein.

Another approach uses recombinant bacteriophage to produce large libraries. Using the “phage method” (Scott and Smith, Science 1990, 249:386-390; Cwirla, et al., Proc. Natl. Acad. Sci. USA 1990, 87:6378-6382; Devlin et al., Science 1990, 49:404-406), very large libraries can be constructed (106-108 chemical entities). A second approach uses primarily chemical methods, of which the Geysen method (Geysen et al., Molecular Immunology 1986, 23:709-715; Geysen et al. J. Immunologic Methods 1987, 102:259-274; and the method of Fodor et al. (Science 1991, 251:767-773) are examples. Furka et al. (14th International Congress of Biochemistry 1988, Volume #5, Abstract FR:013; Furka, Int. J. Peptide Protein Res. 1991, 37:487-493), Houghton (U.S. Pat. No. 4,631,211) and Rutter et al. (U.S. Pat. No. 5,010,175) describe methods to produce a mixture of peptides that can be tested as NF-κB modulators.

In another aspect, synthetic libraries (Needels et al., Proc. Natl. Acad. Sci. USA 1993, 90:10700-4; Ohlmeyer et al., Proc. Natl. Acad. Sci. USA 1993, 90:10922-10926; Lam et al., PCT Publication No. WO 92/00252; Kocis et al., PCT Publication No. WO 9428028) and the like can be used to screen for compounds according to the present invention.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., TIBTech 1996, 14:60).

Classes of compounds that may be identified by such screening assays include, but are not limited to, small molecules (e.g., organic or inorganic molecules which are less than about 2kd in molecular weight, are more preferably less than about 1 kD in molecular weight, and/or are able to cross the blood-brain barrier or gain entry into an appropriate cell, as well as macromolecules (e.g., molecules greater than about 2kD in molecular weight). Compounds identified by these screening assays may also include peptides and polypeptides. For example, soluble peptides, fusion peptides members of combinatorial libraries (such as ones described by Lam et al., Nature 1991, 354:82-84; and by Houghten et al., Nature 1991, 354-84-86); members of libraries derived by combinatorial chemistry, such as molecular libraries of D- and/or L-configuration amino acids; phosphopeptides, such as members of random or partially degenerate, directed phosphopeptide libraries (see, e.g., Songyang et al., Cell 1993, 72:767-778); antibodies, including but not limited to polyclonal, monoclonal, humanized, anti-idiotypic, chimeric, or single chain antibodies; antibody fragments, including but not limited to FAb, F(ab′)₂, FAb expression library fragments and epitope-binding fragments thereof.

The compounds used in such screening assays are also preferably essential pure and free of contaminants that may, themselves, alter or influence gene expression. Compound purity may be assessed by any number of means that are routine in the art, such as LC-MS and NMR spectroscopy. Libraries of test compounds are also preferably biased by using computational selection methods that are routine in the art. Tools for such computational selection, such as Pipeline Pilot™ (Scitegic Inc., San Diego, Calif.) are commercially available. The compounds may be assessed using rules such as the “Lipinski criteria” (see, Lipinski et al., Adv. Drug Deliv. Rev. 2001, 46:3-26) and/or an other criteria or metrics commonly used in the arts.

### Antibodies

Antibodies, or agents comprising the antigen-binding portion of such an antibody, directed against uPA, uPAR, plasmin, plasminogen, or PAI-1 are among the inhibitors useful for the treatment of RA and other diseases and conditions characterized by degeneration of extracellular matrix components.

The drug target proteins or derivatives or analogs thereof, including fusion proteins, may be used as immunogens to generate antibodies that recognize the native protein. Such antibodies include, but are not limited to, polyclonal, monoclonal, humanized monoclonal, chimeric, single chain, Fab fragments, and a Fab expression library, prepared according to known and well-established methods. Such an antibody is preferably specific for (i.e., specifically binds to) and inhibits human or murine uPA, uPAR, plasmin, plasminogen, or PAI-1. Various antibodies directed to these proteins are commercially available, e.g., from American Diagnostica Inc. (Greenwich, Conn.), and Biopool (Umeå, Sweden).

To prepare polyclonal antibodies, purified human glu-plasminogen can be bought from Biopool, or purified from human plasma using gel filtration chromatography, administered to rabbits, and the resulting IgG purified by chromatography on protein-A—Sepharose (Pharmacia Biotech, Sweden). To enhance the immunogenic response, the protein or derivative thereof can be conjugated to an immunogenic carrier, e.g., bovine serum albumin (BSA) or keyhole limpet hemocyanin (KLH), administered together with an adjuvant such as Freund's (complete and incomplete).

For preparation of monoclonal antibodies directed toward a protein in the plasminogen-activation pathway, or a fragment, analog, or derivative thereof, the hybridoma technique originally developed by Köhler and Milstein (Nature 1975, 256:495-497), as well as the trioma technique, the human B-cell hybridoma technique (Kozbor et al., Immunology Today 1983;4:72; Cote et al., Proc Natl Acad Sci U.S.A.; 80:2026-2030, 1983), and the EBV-hybridoma technique to produce human monoclonal antibodies (Cole et al., In: Monoclonal Antibodies and Cancer Therapy, Alan R. Liss, Inc., 77-96; 1985) can be used. Monoclonal antibodies can also be produced in germ-free animals (WO 89/12690), or as “chimeric” or “humanized” antibodies (Morrison et al., J. Bacteriol. 1984;159:870; Neuberger et al., Nature 1984;312:604-608; and Takeda et al., Nature 1985;314:452-454). Such human or humanized chimeric antibodies are preferred for use in therapy of human diseases or disorders (described infra), since the human or humanized antibodies are much less likely than xenogenic antibodies to induce an immune response, in particular an allergic response.

In a preferred embodiment, antibodies that antagonize the activity of uPA, plasmin, plasminogen, and/or PAI-1 are generated. For example, intracellular single chain Fv antibodies can be used to regulate inhibit activity or the selected protein (Marasco et al., Proc. Natl. Acad. Sci. U.S.A. 1993;90:7889-7893; Chen, Mol. Med. Today; 3:160-167; 1997; Spitz et al., Anticancer Res. 1996;16:3415-22; Indolfi et al., Nat. Med. 1996;2:634-635; and Kijma et al., Pharmacol. Ther. 1995;68:247-2675). Such antibodies can be tested using the assays for identifying and evaluating drug candidates.

### Compositions and Formulations

The substances or compounds identified by the methods described herein, including small synthetic compounds, naturally occurring compounds, polypeptides, nucleic acid molecules, and antibodies of the invention, may be used for modulating the biological activity of a drug target, and they may be used in the treatment of arthritis.

Accordingly, the substances (inhibitors, antibodies, drugs, and compounds) may be formulated into pharmaceutical compositions for administration to subjects of a therapeutic amount in a biologically compatible form suitable for administration in vivo. The active substance may be administered in a convenient manner such as by injection (subcutaneous, intravenous, etc.), oral administration, inhalation, transdermal application, or rectal administration. Depending on the route of administration, the active substance may be coated in a material to protect the compound from the action of enzymes, acids and other natural conditions that may inactivate the compound.

The compositions described herein can be prepared by methods known per se for the preparation of pharmaceutically acceptable compositions which can be administered to subjects (for example, see Remington's Pharmaceutical Sciences (Mack Publishing Company, Easton, Pa., USA 1985)). After pharmaceutical compositions have been prepared, they can be placed in an appropriate container and labeled for treatment of an indicated condition. For administration of an inhibitor of a polypeptide of the invention, such labeling would include amount, frequency, and method of administration. The active agent may be in a solid (e.g., capsule, tablet, powder) or liquid (e.g., solution, suspension, emulsion) dosage form and may be administered with pharmaceutical acceptable excipients and fillers, well known to those skilled in the art.

The term “effective amount” of an active agent refers to a nontoxic but sufficient amount of a compound to provide the desired local effect and performance at a reasonable benefit/risk ratio attending any medical treatment. The effective amount of a compound can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The efficacy and toxicity of a compound can be determined by standard pharmaceutical procedures in cell cultures or experimental animals, e.g., ED₅₀ (the dose leading to the desired effect in 50% of the population) and LD₅₀ (the dose lethal to 50% of the population). A pharmaceutically useful dosage lies preferably within a range that includes the ED₅₀ with little or no toxicity. The dosage varies depending upon the disease or condition to be treated or prevented, dosage form employed, sensitivity of the patient, and the route of administration. The exact dosage is chosen by the individual physician in view of the patient to be treated.

### Treatment of Arthritis

The present invention also relates to methods for treating various conditions characterized by destruction of extracellular matrix structures, particularly bone and cartilage, more particularly joints. The invention thus provides methods to interfere with the development of conditions such as arthritis in humans by inhibiting the activity or formation of uPA, uPAR, PAI-1 or plasminogen/plasmin. The inhibitor may be a direct or indirect inhibitor prepared and identified according to the methods described infra.

The proteins uPA and plasmin are proteases that can be inhibited by protease inhibitors. For example, the protease inhibitor Trasylol (aprotinin), a natural proteinase inhibitor that can be obtained from bovine lung, is known to efficiently inhibit plasmin, and may therefore be administered to mammals (including humans) for the treatment or prevention of RA or other arthritic conditions. Trasylol has a molecular weight of 6512 D, and comprises 58 amino acid residues. In addition, as described above, α2-antiplasmin (α2AP), first isolated from human plasma, is an important inhibitor of plasmin in the circulatory system. This protein can be isolated from human plasma, and purified preparations of α2AP is commercially available (e.g., Biopool, Umeå, Sweden).

In an alternative embodiment, the composition comprises other synthetic molecules or small molecules as such as amiloride, 1-anilinonaphthalene-8-sulfonic acid, or analogs or derivatives of these compounds that can safely be administered to patients. Amiloride is an inhibitor of uPA. The activity inhibitor can also be an antibody, directed against either one of the target proteins, which inhibits or reduces the activity of the component upon binding. Furthermore, any one of the uPA, plasminogen, uPAR, and PAI-1 proteins can be inhibited by preventing their expression, translation, or post-translational processing using indirect inhibitors.

Thus, various biologically active compounds that act as inhibitors of plasmin, uPA, uPAR, PAI-1 or plasminogen formation or activation, and which may be used to treat RA in mammals including humans are: uPA inhibitors, including monoclonal or humanized monoclonal antibodies against uPA, and amiloride; plasmin or plasminogen inhibitors including Trasylol and monoclonal or humanized monoclonal antibodies against plasmin and/or plasminogen; and PAI-1 inhibitors including monoclonal or humanized monoclonal antibodies against PAI-1 and 1-anilinonaphtalene-8-sulfonic acid. For treatment or prevention of arthritis, preferred inhibitors include Trasylol, low molecular weight inhibitors to uPA and plasmin, and humanized antibodies or synthesized peptides directed against against uPA, plasmin, PAI-1 or UPAR. These inhibitors can be administered to mammals (including humans afflicted with or at risk for arthritis, particularly RA, in order to treat this disease.

In a preferred embodiment for monoclonal antibodies or humanized antibodies that inhibit the drug targets of the invention, between 0.5 and 10 mg, preferably between 1 and 3 mg, per kilo of body weight per 2-12 weeks, preferably about 8 weeks interval of day of is administered intravenously to a patient afflicted with arthritis, particularly rheumatoid arthritis. Alternatively, a lower dose of antibody could be administered intraarticularly in a sustained release form.

In another embodiment, Trasylol is used to treat or prevent arthritis. Trasylol is commercially available from Bayer (Germany) at a concentration of 1.4 mg/ml or 10,000 KIU/ml in 0,9% NaCl. This solution can be administered locally, for example by intraarticularly injection in the arthritic joints, at a dosage of 1-50 ml/hour, preferably 2-10 ml/hour for a suitable period of time, e.g., for 15 minutes to 2 hours, preferably 30 minutes, per day. The treatment can continue for 2-3 weeks and the treatment effect thereafter evaluated by routine clinical examination. All patients should receive a test dose (1 ml/10 minutes) intravenously before the treatment is started.

In yet another embodiment, α2AP is used for preventing or treating arthritis. Preferably, a sterile solution of the protein is injected intraarticularly into the arthritic joint to locally bind plasmin. The dosage can be from 0.1 mg/ml to 4 mg/ml per joint, daily for 1 to 8 weeks, preferably about 3 weeks, and more preferably from about 0.2 mg/ml to 2 mg/ml per joint daily for 3 weeks. After this period, alleviation of arthritis can be evaluated, and additional α2AP administered, if necessary.

Drugs such as humanized monoclonal antibodies against inflammatory cytokines which down-regulate the expression of uPA, uPAR, plasminogen and/or PAI-1 can administered to patients afflicted with arthritis, particularly RA, by administering between about 0.5 and 10 mg, preferably between 1 and 3 mg, per kilo of body weight per 2-12 weeks, preferably about 8 weeks interval of day of humanized antibody is administered intravenously to a patient afflicted with arthritis, particularly rheumatoid arthritis. Alternatively, a lower dose of antibody could be administered intraarterially in a sustained release form.

### EXAMPLES

The invention is illustrated in the following examples, which are provided by way of illustration and are not intended to be limiting.

### Example 1

### CIA Susceptible Mice Lacking Gene for uPA or Plasminogen

In order to study the functional role of uPA in CIA, the uPA deficiency was crossed into the arthritis susceptible mouse strain (DBA/1) that develops arthritis following injections with collagen type II. The data from the study shows that wild-type mice have a higher incidence of CIA and develop more severe arthritis than the uPA deficient mice. These results are in contrast to those presented by Busso et al. (J. Clin. Invest; 1998, 102:41-50) who showed that arthritis was exacerbated in mice lacking uPA.

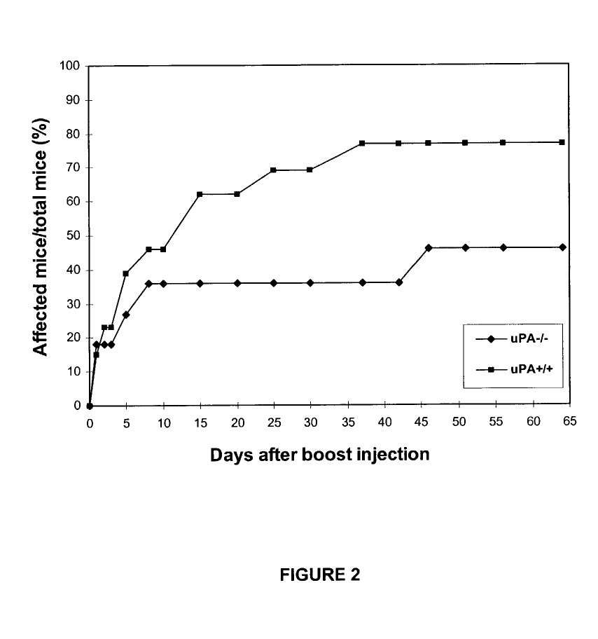

The data obtained suggests that activation of plasminogen by uPA may play a pivotal role in the development of CIA in mice. Therefore, similar studies were carried out in arthritis susceptible plasminogen deficient mice and matched wild-type controls. The results revealed that none of more than 50 plasminogen deficient mice developed arthritis within a 2 month period while more than 75% of the wild-type mice developed the disease (see FIG. 2). FACS analysis revealed that the deficient mice had the MHC-II Aq surface molecule, which determines the susceptibility to CIA. The anti-CII antibody levels in collagen type-II immunized wild type and plasminogen deficient mice were also similar, indicating that both genotypes had a similar immune response.

Finally, the wild-type plasminogen phenotype was reconstituted in plasminogen deficient DBA/1 mice by intravenous injection of 1 mg of human plasminogen every 24 hours. Arthritis was induced by injection of two monoclonal antibodies against collagen type II.

### Methods

Animals. Plg-deficient mice, backcrossed 6 times to C57BL/6 background, were crossed two times to DBA1/J background containing the H-2q MHC class II alleles which mediate their susceptibility to CIA. Through subsequent intercrossing of mice that were heterozygous for the Plg-deficiency, we obtained wildtype (plg+/+), heterozygous (plg^{+/-}) and homozygous (plg^{-/-}) mice that were subsequently used in the experiments. uPA deficient mice backcrossed 6 times to C57B1/6 background were crossed once to DBA1/J background. The heterozygous litters were used in breedings. The wildtype (uPA^{+/+}), heterozygous (uPA^{+/-}) and homozygous (uPA^{-/-}) offspring from these breedings were used in CIA experiments. Only male siblings were used in the experiments. All of the mice used were genotyped for the H-2q MHC class II alleles, which mediates the susceptibility to CIA.

Genotyping of the animals. Genomic DNA was isolated from mouse tail tips and genotyped by PCR. The sequences of the primer pairs used in the PCR reaction were as follows:

uPA:

SEQ ID NO:11

5′ ATC GAA GGC CGC CCA ACT CTG AGT GGG ATT G 3′

SEQ ID NO:12

5′ TCC CAA CAG CAG ATC TCA TGA ATG ACC C 3′
neo:

SEQ ID NO:13

5′ ATG ATT GAA CAA GAT GGA TTG CAC G 3′

SEQ ID NO:14

5′ TTC GTC CAG ATC ATC CTG ATC GAC 3′
plg:

SEQ ID NO:15

5′ TCA BCA GGG CAA TGT CAC GG 3′

SEQ ID NO:16

5′ CTC TCT GTC TGC CTT CCA TGG 3′
H-2q:

SEQ ID NO:17

5′ CCG CAG GGA GGT GTG GGT 3′

SEQ ID NO:18

5′ ATT TCG TGG CCC AGT TGA 3′

Induction of collagen-II induced arthritis. Collagen-induced arthritis (CIA) was induced in mice with Rat collagen II, which was prepared from the Swarm chondrosarcoma after pepsin digestion (Andersson and Holmdahl, Eur. J. Immunol.;20:1061-1066, 1990). Collagen II was dissolved at a concentration of 2 mg/ml in 0,1 M acetic acid and stored at 4 degrees. Arthritis was induced by intrademal injection at the basis of the tail with 100 ml of 100 mg rat collagen II emulsified with an equal volume of complete Freund's adjuvant (CFA, with Mycobacterium butyricum; Difco, Detroit, Mich.). 21 days later, mice were boost injected again with 50 ul of 50 mg rate collagen II emulsified with an equal volume of incomplete Freund's adjuvant (IFA, Difco). Experimental protocols were approved by the Regional Ethical Committee of Umea University.

Induction of arthritis using monoclonal antibodies directed against collagen type II. A battery of monoclonal antibodies against collagen type II were produced by standard procedures. Induction of arthritis was performed by intravenous injection of a cocktail of two antibodies denoted C1 and M2139 at day 0. Five days later (day 5), lipopolysacchride was injected intraperitoneally to enhance the immune response. The development of arthritis was evaluated with a previously described clinical grading system.

Clinical grading evaluation of arthritis. The development of arthritis was tracked using a scoring system which defines one inflamed toe or knuckle as 1 point and one inflamed wrist or ankle as 5 points, resulting in a score of 0-15 points for each paw and 0-60 points per mouse. Deformed or swollen without redness is not included in this system.

Morphological staining of arthritis. At the end of the experiment, the mice were sacrificed after which wrist and paw joints were dissected and fixed in 4% phosphate buffered paraformaldehyde solution at 4° C. for 24 hours. The samples were then decalcified in 10% EDTA for 3 weeks before being embedded in paraffin. 8 mm sections were stained either with hematoxylin and erythrosin or fast green and Safranin O.

Quantification of anti-collagen II specific antibody levels in serum. Mice were tail-bled and the individual serum samples were collected and stored at −80° C. until assayed. 96-well ELISA plates (Costar, Cambridge, Mass.) were coated overnight at 4° with 50 ml/well of PBS containing 10 mg/ml of native rat collagen II for the quantification of anti-collagen II auto-antibodies in sera. All tests were carried out in duplicate. Washings were performed using Tris-buffered saline (pH 7.4) containing 0.1% Tween 20. The amount of bound antibody was estimated after incubation with either a sheep anti-mouse IgG mAb or a goat anti-mouse IgM mAb, both coupled to alkaline phosphatase (Jackson ImmunoResearch, West Grove, Pa.). The subsequent quantification of bound enzyme was performed with paranitrophenol as a chromogenic substrate and the absorbance was determined in a Titertek multiscan spectrophotometer. The amount of CII-specific antibodies in sera, from immunized mice was determined by comparing the titration curve of the test serum with the titration curve of a standard consisting of affinity-purified collagen II reactive antibodies.

Immuno-histochemistry analysis of the joints. Front paws were demineralized in 10% EDTA without any previous fixation, and subsequently snap-frozen in isopentane, prechilled with liquid nitrogen and kept at −70° C. until cryosectioned. 6 to 8 mm sections were cut frontally. All sections were fixed in cold acetone for 5 minutes, washed in PBS (PH 7,4), and depleted for endogenous peroxidase by treatment with 0.3% H₂O₂ for 10 minutes. After additional washes in PBS (pH 7.4), the sections were incubated with rat monoclonal antibody diluted with PBS (pH 7.4) and containing 4% bovine serum albumin. Biotin-labeled rabbit anti-rat immunoglobulins were used as secondary antibodies. Binding of biotin-labeled antibodies was detected with a rat ABC staining system (Santa Cruz, Calif.). All sections were counter-stained with Mayer's hematoxylin.

Flow cytometry analysis. For staining of fresh peripheral blood leukocytes, ammonium chloride (0.84%, pH 7.4) was added to the blood, for 3 min, in order to lyse the red blood cells. Cells were then washed and re-suspended in PBS supplemented with 0.5% BSA. Cells were stained with 20 μl of staining buffer containing 0.5 μg of antibody against H-2q surface molecule. Samples were washed in staining buffer, fixed in buffered 1% paraformaldehyde and stored in the dark at 4° C. until analyzed.

Induction of arthritis in plasminogen deficient mice by restoration of plasminogen. 100 μl of human plasminogen, at a concentration of 10 mg/ml, was injected intravenously into the plasminogen deficient mice every 24 hours in order to restore the serum plasminogen level. 12 hours after the first injection of plasminogen, three groups of mice, plg-wildtype, plg-heterozygous, plg-deficient were again injected with plasminogen before arthritis was induced using the Collagen II monoclonal antibody cocktails as described above. The development of arthritis was followed by the clinical scoring system.

Statistical analyses. The incidence of arthritis was analyzed by proportionate group frequencies. The Mann Whitney U-test was used for analysis of arthritic scores and the onset. Antibody levels were analyzed by the two-tailed unpaired t-test with P<0,05 considered as significant. The results of this test can establish that the presence of PA increases the risk that a mammal (including humans) will develop arthritis.

### Results

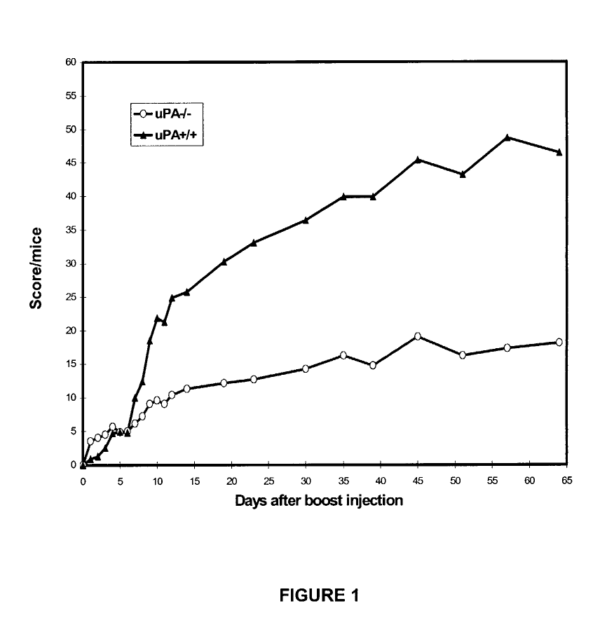

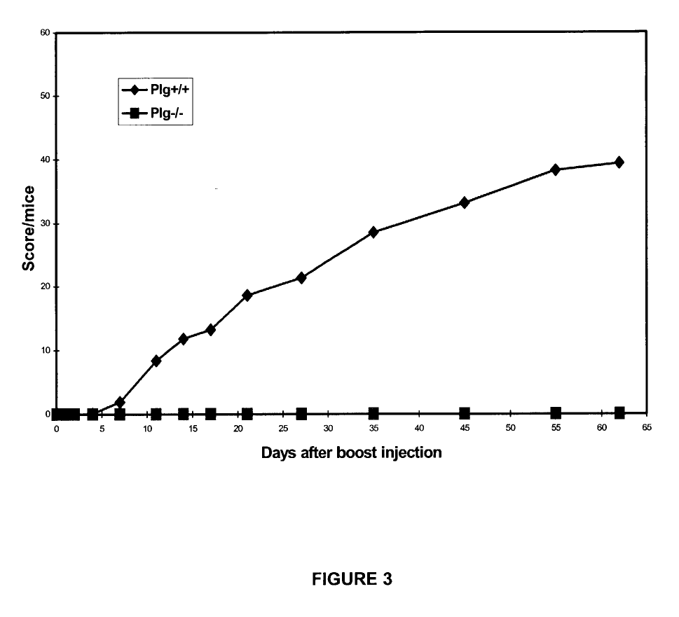

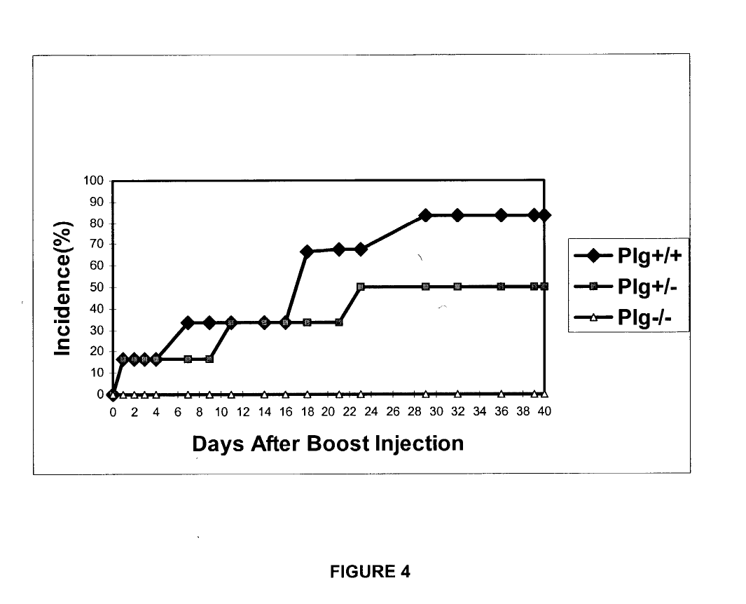

Macroscopic evaluation of the collagen-induced arthritis in uPA and plg mice. The progeny from uPA^{+/-}X uPA^{+/-}and plg^{+/-}X plg^{+/-}breedings, wildtype (uPA^{+/+}, plg^{+/+}), heterozygous (uPA^{+/-}, plg^{+/-}) and homozygous (uPA^{-/-}, plg^{-/-}) were used for the experiments. For the progeny from the uPA-breedings, the wild-type mice had higher arthritic scores at day 5, as compared to homozygous mice, and from day 10 and onwards, the difference was significant. (P<0.05; FIGS. 1 and 2). The wild-type mice also showed significantly higher incidence compared to the uPA deficiency mice (P<0.05). Incidence at day of onset and mean arthritic score are shown in Table 2. No difference in incidence, onset day and maximum score could be seen between either the wild-type and heterozygous, or the heterozygous and knockout mice groups. However, when the same experiments were performed on plg-deficient mice, none out of 30 deficient mice developed arthritis. Additionally, the plg-heterozygous mice developed a significantly lower incidence of arthritis as compared to the wild-type siblings (P<0.05, FIG. 3). The incidence of the disease also indicates that it is significantly lower in pig heterozygous mice (P<0.05) (see FIG. 4). As the pig heterozygous mice have half of the amount of plasminogen in their bodies, the development of CIA may be dose dependent. Moreover, a significantly delayed onset also was observed on these mice (Table 3), which shows that plasminogen is involved in the initiation of the disease.

Morphology of uPA mice after induction of CIA. At the conclusion of the experiment, the mice hind paws were taken for morphological analysis. For uPA wildtype and deficiency paws with the same clinical scores, there were no substantial morphological differences. However in uPA deficient affected joints, fibrin deposition and synovial hyperplasia could be seen in the marginal zone at the early stage. Later, the mononuclear cells became the main cell type to infiltrate into the synovial cavity, and the cartilage itself showed severe degradation. Lastly, fibrotic synovium was viewed with pannus. Severe destruction of cartilage and underlying bone with newly formed cartilage and bone tissues were also presented.

Morphology of plasminogen mice after induction of CIA. Morphological evaluation showed that after injection of collagen type II at the basis of the tail, the plg deficient mice have normal joint morphology with intact cartilage and no inflammation of peripheral synovial tissue, indicating that the plasminogen deficient mice are totally resistant to the disease. The plg heterozygous affected mice have a similar proliferation of connective tissue (fibroblasts) in the synovium. Adjacent tissue, cartilage and bone also were degraded and in the late stage new bone and cartilage formation could be seen. These morphological changes are similar, compared to the wildtype mice. The maximum scores of heterozygous mice showed no difference from the wildtype maximum scores.

No difference was found between the collagen-II antibody titers in plasminogen wild-type and deficient mice. Since the plasminogen deficient mice could not be induced by normal CIA during our experiments, we investigated if such null response toward collagen II challenge was the result of immune defects. 60 days after boost injection, the sera of experiment 1 were taken from the eyes and anti-collagen II antibody ELISA was performed on these sera. The collagen II specific antibody response was normal in the plasminogen deficient mice compared to wild-type mice, although they had significantly different clinical scores. This indicates that the plasminogen deficient mice had a normal antibody production pathway.

Antibodies against collagen type II have a normal binding to collagen type II in plasminogen deficient mice. In order to confirm that monoclonal antibodies against type II collagen bind equally well to collagen type II in both wild type and plasminogen deficient mice, biotinolayted anti-collagen type II antibody was injected intraperitoneally into neonatal wild type or plasminogen deficient mice. 24 hours later, the mice joints were dissected and performed for immunohistochemistry for the anti-collagen II antibody binding. The results showed that there was similar antibody binding at the surface of the cartilage in wild type and plasminogen deficient mice. However there were no macrophages in the synovial space in the plasminogen deficient mice that were immunized. The titer of antibodies against collagen type II was high in both wild-type and plasminogen deficient mice during CIA but there was no inflammatory response in the plasminogen deficient mice. The data shows that plasminogen plays a role during CIA at a stage after the antibody binding, possibly at the stage of macrophage activation and/or activation of the complement system.

Macroscopic evaluation of monoclonal antibody against collagen II induced arthritis in uPA and plg mice. The wildtype (uPA^{+/+}, plg^{+/+}), heterozygous (uPA^{+/-}, plg^{+/-}) and homozygous (uPA^{-/-}, plg^{-/-}) siblings with the same background compared to the mice used in CIA were used for the experiments. With uPA mice, the uPA deficient mice developed the most severe arthritis during the first 10 days, while the uPA wildtype and heterozygous mice similarly had less severe arthritis. After day 10, the uPA deficient mice quickly underwent subsidence of severity compared to uPA wildtype and heterozygous mice. uPA wildtype and heterozygous mice kept similar arthritis levels until day 28 and subsequently the heterozygous mice developed less arthritis than wild-type, but still of higher severity than the deficient mice. In plasminogen mice, the wild-type mice had the most severe arthritis during the 45 days entire period with intense subsidence after 32 days. The severity of heterozygous mice always was less in comparison to wild-type, while in contrast, the plasminogen deficient mice did not have any inflammation during the disease process indicating again that plasminogen plays an essential role for the development of experimental arthritis in mice. Thus, inhibition of plasminogen can be used to treat or prevent RA in mammals including humans. The morphology of plasminogen and uPA mice induced with antibodies against collagen II was also studied.

I.V. administration of plasminogen into plasminogen deficient mice converted its phenotype. It was desired to confirm the results finding that plasminogen deficient mice never developed arthritis induced by either by type II collagen immunization or anti collagen II monoclonal cocktails. Human plasminogen was injected into plasminogen deficient mice and 12 hours later challenged with collagen II monoclonal cocktails. Five days after the cocktail injection, 3 out of 4 plasminogen deficient mice did develop joint inflammation, as well as 3 out of 7 heterozygous mice and all of the 5 wild-type mice. In contrast, plasminogen deficient mice without treatment developed no signs of inflammation. The phenotype of plasminogen deficient mice during monoclonal antibody induced arthritis was confirmed.

Migration of inflammatory cells is impaired in plasminogen deficient mice after induction of arthritis. In order to determine if the plasminogen deficient mice have impaired inflammatory cell migration during Collagen II induced arthritis, immunohistochemistry was performed on sections of joints from plasminogen wildtype and plasminogen deficient mice that were immunized with Collagen II. Immunohistochemical staining of macrophages using a macrophage-binding antibody was conducted of a section of a joint from a wild type control mouse with arthritis at 40 days after boost injection. The stained sections showed an extensive influx of macrophages at the border of destructed tissue, with large numbers of macrophages migrating into the joint space in the wild type mice that had severe arthritis, especially at the frontier of the invading pannus. Immunohistochemical staining of macrophages in a section of a joint from a plasminogen deficient mouse stained for macrophages showed only very few resting macrophages and no tissue destruction.

### Discussion

Collagen arthritis is a model for autoimmune arthritis. The results described above showed that uPA and plasmin play pivotal roles in collagen II induced arthritis or in arthritis induced by administration of a monoclonal antibody cocktail against mice type II collagen.

One of the goals was to treat mice of different genotypes with a CIA model and analyze the influence of plasminogen on disease development. Collagen II induced arthritis can only be induced on the mouse strains with certain major histocompatibility complex (MHC) halotypes (for instance, DBA/1J mice express H-2q). C57/B6 mice express the H-2b halotype and therefore they are resistant to the disease (Holmdahl et al., Immunogenetics 1986;24:84-89). The study was therefore initiated by intercrossing plasminogen deficient mice with 8 times backcrossed into C57/B6 into CIA susceptible strain, DBA1/J. The plasminogen gene is located on 7.3 cM of chromosome 17 only 10 cM away from MHC-II clusters which determine the susceptibility. Thus there is a possibility that when the intercrossing was made, recombination took place on the MHC region, which destroyed the expression of MHC II and therefore those mice did not have the ability to present the collagen type II antigen. The expression of H-2q molecule expression on the leukocytes was also checked, showing a positive expression of q molecule, thus excluding this possibility.

Since the antibody level against CII is directly related to the susceptibility, the antibody level in the serum was measured in some plasminogen wild type and deficient mice. There was no difference between the two groups, thus implying that the humoral immune response was normal in plasminogen deficient mice. Other studies have implied that plasminogen is important in cell migration (Jackson and Reidy, Ann NY Acad Sci 1992;667: 141-150). Bordetella pertussis was therefore injected into the mice intraperatoneally to determine if, by enhancing the permeability of the vessels, the plasminogen deficient mice could develop the disease. The results showed that with this injection, heterozygous mice had similar levels of disease as compared to wild-type, whereas the plasminogen deficient mice still could not develop any inflammation, suggesting that vessel permeability was an important, but not essential, reason for the inflammatory cell migration.

To distinguish whether plasminogen was involved in immune response or in the effect stage, monoclonal antibody cocktails were injected intravenously into the mice (Holmdahl et al., Arthritis Rheum 1986;29:400-410). The data showed that with the injection, plasminogen wild-type still exhibited the most severe disease, heterozygous mice had the middle level of severity, while no plasminogen deficient mice developed any signs of inflammation.

These results show that plasminogen plays an important role during the effect stage of the disease. A plasminogen restoration experiment on plasminogen deficient mice confirmed this. During CIA, migration of inflammatory cells is triggered by the immune system. This indicates that plasminogen plays a role in the mechanisms connecting the activated immune system with inflammatory cell migration.

In CIA experiments using uPA wild-type and uPA deficient mice, the uPA deficient mice developed more severe arthritis compared to wild-type and heterozygous mice during the first 5 days. Subsequently, however, this trend shifted and uPA deficient mice had less arthritis compared to wild-type and heterozygous mice, while uPA wildtype had higher incidence of arthritis than the heterozygous mice.

In conclusion, the results demonstrate that uPA and plasminogen are critical for the pathogenesis of CIA through PA system mediated tissue destruction.

TABLE 2
Mice deficient for uPA are less prone to CIA

| | | Onset day | Arthritic score |
|---|---|---|---|
| Genotypes | Incidence | (mean ± SD) | (mean ± SD) |
| uPA +/+ | 41/48 | 35.3 ± 14.9 | 38.2 ± 18.6 |
| uPA +/− | 20/22 | 35.8 ± 13.5 | 29.8 ± 19.3 |
| uPA −/− | 28/49 | 44.5 ± 17.7 | 24.3 ± 15.7 |

The results in Table 1 show that out of 49 mice deficient for uPA, only 28 developed CIA.

TABLE 3
Plasminogen deficient mice are resistant to CIA

| | | Onset day | Arthritic score |
|---|---|---|---|
| Genotypes | Incidence | (mean ± SD) | (mean ± SD) |
| plg +/+ | 25/30 | 40.0 ± 14.94 | 39 ± 18.4 |
| plg +/− | 13/19 | 45.8 ± 14.2 | 35 ± 18.7 |
| plg −/− | 0/50 | 0 | 0 |

The results in Table 2 show that none of the plasminogen deficient mice developed CIA.

TABLE 4
Injection of plasminogen restored the arthritis in plasminogen deficient
mice

| Genotype | Incidence of RA | Arthritic score |
|---|---|---|
| plg ^{+/+} | 4/5 | 15.0 ± 7.2 |
| plg ^{+/-} | 3/7 | 12.0 ± 8.8 |
| plg ^{-/-} | 0/5 | 0 |
| plg ^{-/-} with plg | 3/4 | 6.7 ± 2.9 |

### Example 2

### CIA Susceptible Mice Lacking Gene for PAI-1

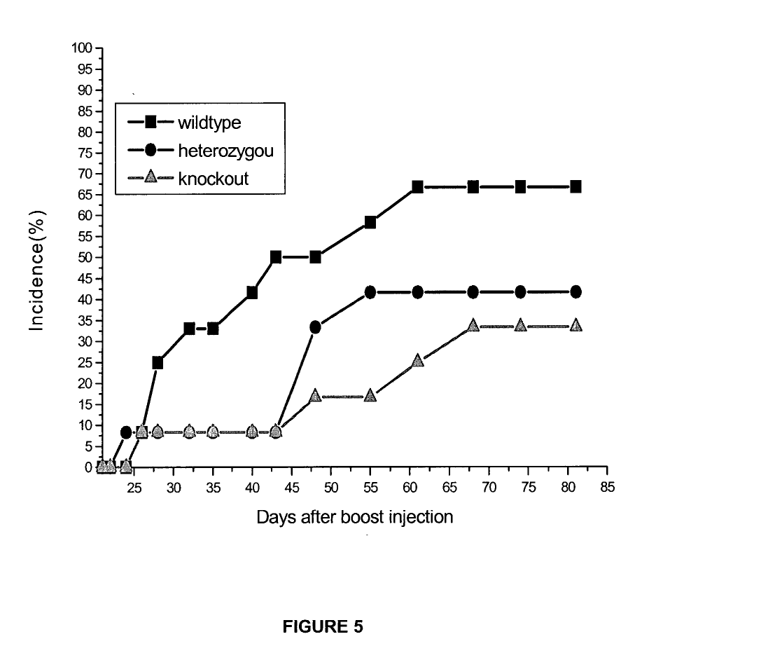

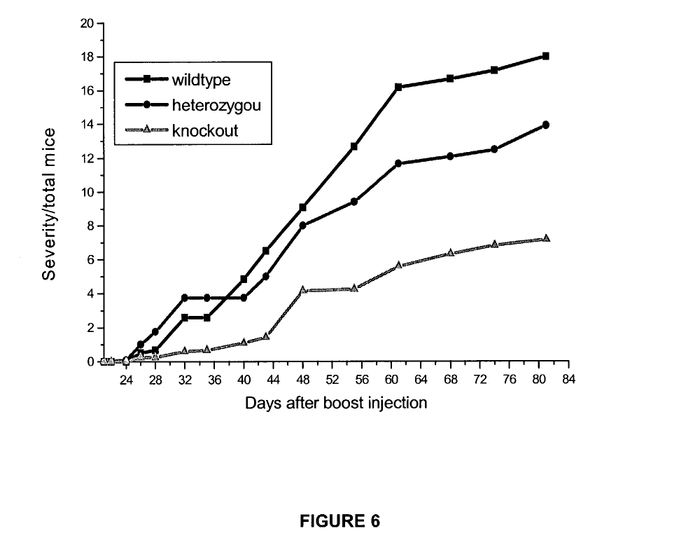

This experiment was performed as the experiment described in Example 1, except that PAI-1 knockout mice were used instead of the uPA knockout mice used in Example 1. The results of this experiment are shown in FIGS. 5 and 6. FIG. 5 shows reduced incidence of collagen-induced arthritis in PAI-1 heterozygous or knock-out mice as compared to wild-type mice. FIG. 6 shows the severity of the arthritis in the mice that were studied.

### Example 3

### The Function Role of tPA During CIA

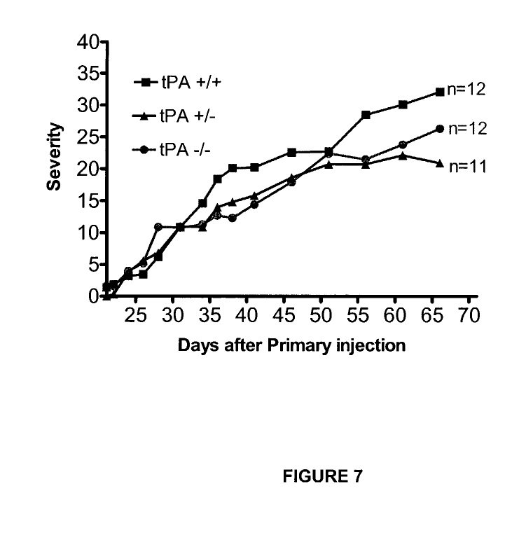

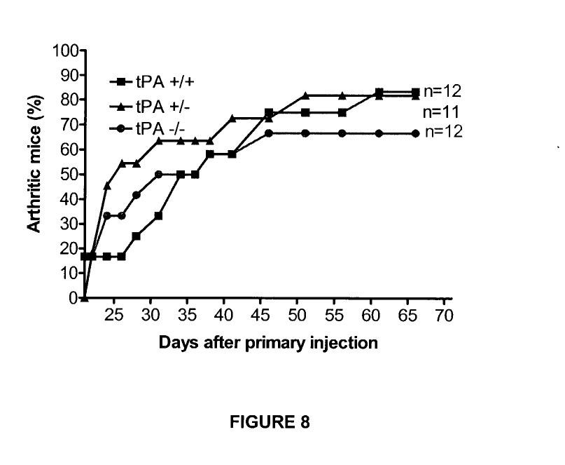

This experiment was performed to investigate the functional role of tPA during CIA. tPA activates plasminogen to plasmin and therefore could be involved in the development of CIA. tPA wild-type and tPA deficient siblings were induced with arthritis and the development and incidence of CIA was followed. There was no difference in the severity or incidence of arthritis between tPA deficient and wild-type control mice (see FIGS. 7 and 8). Thus unlike uPA, tPA does not seem to play any significant role in the development of CIA.

### Example 4

### The Functional Role of the uPA-receptor During CIA

Several different in vivo functional roles have been suggested for uPAR , including focusing plasmin-mediated pericellular proteolysis to the cell surface as well as regulating cell adhesion and cell migration in both a proteolytic and non-proteolytic fashion.

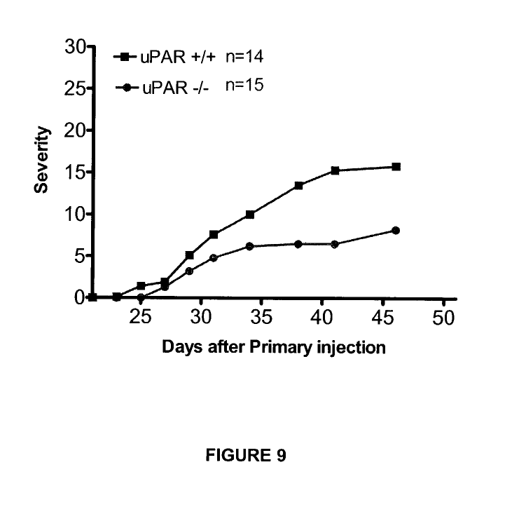

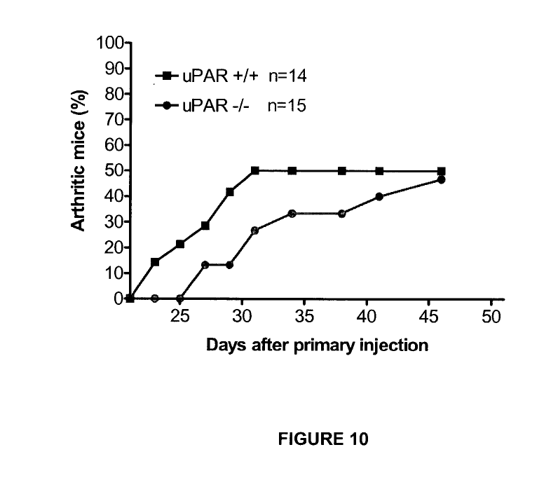

This experiment was conducted to determine the role of uPAR during CIA by using uPAR deficient mice and wild-type control mice. The wild-type and deficient mice were induced with arthritis and the development and incidence of CIA was followed. Both the severity and the incidence of arthritis were lower in the uPAR deficient mice (see FIGS. 9 and 10). Thus uPAR is involved in promoting the development of CIA.

### Example 5

### In Vitro Screening Assay for uPA Inhibitors

To investigate uPA inhibitor activity, a chromogen assay is used that is based on the difference in absorbance (optical density) between the product (pNA) formed and the original substrate (S-2444; Chromogenix-Instrumentation Laboratory SpA, Milano, Italy). S-2444 (L-pyroglutamyl-glycyl-L-arginine-p-nitroaniline hydrochloride, Molecular Weight=499) is a chromogenic substrate for uPA (urokinase). The rate of pNA formation, i.e., the increase in absorbance per second at 405 nm, is proportional to the enzymatic uPA activity and is conveniently determined with a photometer. The Kₘ for human uPA is 9×10⁻⁵ mol/L.

When an inhibitor is added into the system, uPA is inhibited and thus the chromogen cannot be formed. Based on the different amount of formed pNA, the inhibitor activity can be determined.

uPA (urokinase) is diluted from stock (1 mg/ml, Wakamoto Pharmaceutical, Tokyo, Japan) to 0.002 mg/ml in 1M lysine. The inhibitor is diluted to a suitable concentration in 1×PBS. S-2444 is diluted from powder stock to 10 mM in 1×PBS solution.

To each well in a 96 well plate, 100 μl S-2444 and 50 μl inhibitor solution is added, testing one inhibitor per well. Individual inhibitor sample blanks are treated identically to the inhibitor samples with the substitution of inhibitor by 1×PBS. 50 μl uPA is then added to each well. Serial dilutions of known concentrations of uPA, without inhibitor treatment, is used to create an absorbance standard curve. The plate is incubated at 37° C. for 0.5 hours, and absorbance at 405 nm is measured using a plate reader.

The absorbance of each well is then related to the standard curve to identify the degree of inhibition in each well. In the wells characterized by low absorbance, uPA inhibition has occurred. The characteristics of each selected inhibitor, i.e., candidate drug, is then further studied by optimizing, for example, dosage levels and schedule in in vivo models.

### Example 6

### In Vitro Screening Assay for Plasminogen/Plasmin Inhibitors

To investigate plasminogen/plasmin inhibition by test drugs, a chromogen assay based on the difference in absorbance (optical density) between the product (pNA; p-nitroaniline dihydrochloride) formed and the original substrate (S-2551, Chromogenix AB, Mölndal, Sweden) is used. S-2251 (H-D-Valyl-L-leucyl-L-lysine-p-nitroaniline dihydrochloride, Molecular weight 551,5) is a chromogenic substrate for plasmin and activated plasminogen. The rate of pNA formation, i.e., the increase in absorbance per second at 405 nm, is proportional to the enzymatic activity and is conveniently determined with a photometer. The Kₘ for human plasmin is 3×10⁻⁴ mol/L

The substrate is insensitive to uPA. Therefore, excess uPA, i.e., a molar excess of at least 10 times as compared to the required minimum amount of uPA to activate all plasminogen in the well, is used to activate plasminogen into active plasmin, and the formed plasmin can further convert excess S-2251 into chromogen pNA. When an inhibitor is added into the system, plasmin is inhibited and thus the chromogen cannot be formed. Based on the different formed pNA, the inhibitor activity is determined. When S-2251 and plasminogen are in a molar excess of at least 10 times as compared to the minimum amount of uPA needed to activate all plasminogen in the well, this method can be adapted to investigate uPA inhibitor activity.

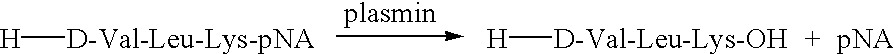

uPA (urokinase) is diluted from stock (1 mg/ml, Wakamoto Pharmaceutical, Tokyo, Japan) to 0.035 mg/ml in 1M lysine. The inhibitor is diluted to a suitable concentration in 1×PBS. S-2251 is diluted from powder stock to 0.2 mM in 1×PBS solution. Plasminogen is diluted to 35 μg/ml in 1×PBS solution.

In a 96 well plate, 160 μl S-2251, 20 μl uPA, and 20 μl inhibitor solution is added to each well. Each inhibitor is added to one (optionally more than one) well Individual inhibitor sample blanks are treated identically to the inhibitor samples with the substitution of inhibitor by 1×PBS. Next, 20 μl plasminogen is added to each well. A series dilution of plasminogen with known concentrations is used for standard curve. The plate is incubated at 37° C. for 4 hours. Absorbance is measured at 405 nm using a plate reader.

The absorbance of each well is then related to the standard curve to identify the degree of inhibition in each well. In the wells characterized by low absorbance, inhibition of plasmin (initially present or formed by converted plasminogen in the presence of uPA) has occurred. The characteristics of each selected inhibitor, i.e., candidate drug, is then further studied by optimizing, for example, dosage levels and schedule in in vivo models.

### Example 7

### In Vitro Screening Assay for uPAR Inhibitors

Mouse LB6 cells, which produce no plasminogen activator, are transfected with a uPAR cloning vector containing the SV40 (human simian virus 40) promoter at the 5′ end and polyadenylation and splice sites at the 3′ end (Okayama and Berg, Mol Cell Biol. 1983;3:280-9). In the presence of uPAR, however, cells bind uPA and hence acquire the ability to degrade casein in the presence of plasminogen (Vassalli et al., J Cell Biol. 1985; 100:86-92).

Since binding is strictly species specific, LB6 cells do not bind human uPA and therefore they will score negative in a caseinolytic plaque assay, even after incubation with human uPA. Expression of human uPAR cDNA by LB6 cells, on the other hand, allow them to bind human uPA and thus to form plaques. The transfected cells can then be subcloned and single clones from each transfection expanded and incubated with uPAR inhibitor. The cells are thereafter rinsed, incubated with human uPA, rinsed again to get rid of excess of uPA, and laid onto a casein plaque assay. The uninhibited uPAR can bind uPA and therefore can degrade the casein plaque. The bound uPA activity is proportional to the area of the caseinolytic plaque.

LB6 cells (2×10⁵) are transfected with 9 μg p-uPAR DNA (Roldan et al., EMBO J. 1990;9(2):467-74) plus 1 μg pRSV neo DNA, using a modification of the calcium phosphate co-precipitation technique (Pozzatti et al., Science. 1986;232:223-7). Cells are plated in 0,8 mg/ml G418 containing DMEM with 10% fetal calf serum, and colonies are isolated after ˜13 days. The pools of transfected clones are tested by the caseinolytic plaque assay (Vassalli et al., Cell 1977; 11:695-705.) and positive clones are picked. After subcloning, several clones from each transfection are tested for human uPA binding, using the same technique. Cells are washed with PBS, incubated in the presence of inhibitor against human uPAR at different dilutions for lh at 37° C. Thereafter, cells are washed again and incubated in the presence of 0.2 nM human uPA for 1 h at 37° C., washed extensively and covered with a thin agar layer containing 1.3% casein, and 17 μg/ml plasminogen. The plates are incubated at 37° C. for 3 h, stained with Coomassie brilliant blue R250, and photographed.

Test agents which are characterized by small or no caseinolytic plaques are uPAR inhibitors. While the assay in itself does not distinguish between uPA inhibitor or uPAR inhibitor, the test agent can be further characterized for uPA inhibition using one of the assays described herein to discriminate between the two types of inhibitors.

### Example 8

### In Vitro Screening Assay for PAI-1 Inhibitors

The assay is performed in microtiter plates. The inhibitor against PAI-1 is measured by an indirect chromogenic assay based on a chromogenic product (pNA) being formed from the a substrate (S-2444; Chromogenix-Instrumentation Laboratory SpA, Milano, Italy) in the presence of active uPA. S-2444 (L-Pyroglutamyl-glycyl-Larginine-p-Nitroaniline hydro-chloride, Molecular Weight=499) is a chromogenic substrate for uPA (urokinase).

The method for the determination of activity is based on the difference in absorbance (optical density) between the pNA formed and the original substrate. The rate of pNA formation, i.e., the increase in absorbance per second at 405 nm, is proportional to the enzymatic activity and is conveniently determined with a photometer. When active PAI-1 is present in the system, it will inhibit the uPA function. Therefore, when an inhibitor against PAI-1 is added in, the inhibition ability of PAI-1 is lost so that uPA can activate the chromogenic reaction.

1. Samples containing PAI-1 and serially diluted candidate inhibitor are diluted in activity assay buffer (0. 15M NaCl, 0.05M Tris-HCl, pH 7.5, containing 0.01% Tween 80 and 100 μg/ml bovine serum albumin), followed by the addition of uPA to 25 uPA IU/ml.

2. Samples (100 μl) are incubated for 30 minutes at 23° C. after which 100 μl of 0.5 mM S-2444 substrate is added.

3. Residual uPA activity is quantitated by measuring the change in absorbance at 405 mn at 5 minutes intervals in a Titertek multiscan spectrophotometer.

The concentration of active PAI-1 is calculated from the amount of sample that inhibits the uPA activity by 50%, as compared to samples containing uPA alone. The activity of PAI-1 inhibitor is calculated from the amount of PAI-1 that inhibits the uPA activity.

### Example 9

### Prevention of Arthritis in Mammals by Administration of Aprotinin

To test potential drug candidates, arthritis is induced in CIA-sensitive mice either by immunization with collagen type II (CIA) or, alternatively, by use of monoclonal antibodies.

Using monoclonal antibodies to induce CIA has an advantage in that it allows for better control of the induction. Arthritis is also induced faster by monoclonal antibodies (arthritic response is detectable after 2 days and maximum effect occurs after about 5-7 days) than with CII, which is an advantage since the animals can be treated with the selected candidate drug for a shorter time.

The present example describes treatment of CIA-sensitive mice with aprotinin (Trasylol; Bayer). Trasylol is an inhibitor of plasmin and thereby, according to the invention, a candidate drug for treating arthritis.

Since aprotinin has a rather short half life in mice, a sustained release system for Trasylol is used. An exemplary system based on water/oil (w/o) emulsions where aprotinin is incorporated is described in detail by Bjerregaard et al. (Journal of Controlled release 2001; 71:87-98).

The mice are injected intraperitoneally with 0.5 mL aprotinin emulsion, containing 30% w/w disperse phase with 87 mg aprotinin per ml aqueous phase (corresponding to a dose of approximately 85,000 KIU (“kallikrein-inactivator units”) aprotinin, using a Hamilton syringe 21 G needle. (It is also possible to carry out controlled administration of aprotinin via an osmotic pump.) The injection is repeated every 72 hours.

CIA induction using monoclonal antibodies. One day after the first injection of aprotinin, induction of arthritis is performed by intravenous injection of a cocktail of two antibodies against collagen type II denoted C1 and M2139 (Holmdahl et al., Autoimmunity 1991;10:27-34). Five days after the induction of antibodies (day 5), lipopolysacchride (“booster”) is injected intraperitoneally to enhance the immune response. The development of arthritis is evaluated with the clinical grading system described in Example 1. Control mice (10 per group) are treated in the same way except that aprotinin is omitted in the water/oil emulsion. The development of arthritis and clinical score in the two groups are compared, showing that the incidence and severity of the CIA is lower in animals treated with aprotinin.

CIA induction using Collagen-II. Collagen-induced arthritis (CIA) is induced in mice with Rat collagen II, which is prepared from the Swarm chondrosarcoma after pepsin digestion, as previously described (Andersson and Holmdahl, Eur J Immunol 1990;20:1061-1066). Collagen II is dissolved at a concentration of 2 mg/ml in 0.1 M acetic acid and stored at 4° C. Arthritis is induced by intrademal injection at the basis of the tail with 100 ml of 100 mg rat collagen II emulsified with an equal volume of complete Freund's adjuvant (CFA, with Mycobacterium butyricum; Difco, Detroit, Mich.). 20 days later, the mice (10 per group) are injected intraperitoneally with 0.5 ml of w/o emulsion containing 30% w/w disperse phase with 87 mg aprotinin per ml aqueous phase (corresponding to a dose of approximately 85,000 KIE aprotinin) and this treatment is repeated every 72 hr. One day after the first injection of the aprotinin emulsion, mice are boost injected again with 50 μl of 50 mg rate collagen II emulsified with an equal volume of incomplete Freund's adjuvant (IFA, Difco). Control mice (10 per group) are treated in the same way except that aprotinin is omitted in the water oil emulsion. The development of arthritis in the two groups is thereafter compared, showing that the incidence and severity of the CIA is lower in animals treated with aprotinin.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference in their entireties.

1. A method of treating or preventing arthritis in a mammal which comprises administering to the mammal an effective amount of an agent that inhibits at least one member selected from the group consisting of plasmin, plasminogen, urokinase-type plasminogen activator, urokinase-type plasminogen activator receptor, and plasminogen-activator inhibitor type 1.

2. The method of claim 1 wherein the agent inhibits plasmin.

3. The method of claim 1 wherein the agent inhibits plasminogen.

4. The method of claim 1 wherein the agent inhibits urokinase type plasminogen activator.

5. The method of claim 1 wherein the agent inhibits urokinase-type plasminogen activator receptor.

6. The method of claim 1 wherein the agent inhibits plasminogen-activator inhibitor type 1.

7. The method of claim 1 wherein the agent comprises a protease inhibitor.

8. The method of claim 7 wherein the protease inhibitor comprises aprotinin.

9. The method of claim 1 wherein the agent comprises amiloride.

10. The method of claim 1 wherein the agent is a monoclonal antibody directed against the at least one member.

11. The method of claim 1 wherein the agent is an anti-sense nucleic acid sequence capable of binding to a nucleic acid encoding the at least one member.

12. The method of claim 1 wherein said arthritis is caused by a degenerative joint disease.

13. The method of claim 1 wherein said arthritis is a member selected from the group consisting of rheumatoid arthritis, psoriatic arthritis, infectious arthritis, juvenile rheumatoid arthritis; osteoarthritis, and spondyloarthropaties.

14. The method of claim 13 wherein said arthritis is rheumatoid arthritis.

15. The method of claim 1 wherein the mammal is a human.

16. A method of screening to identify an agent useful for treating or preventing arthritis which comprises
(i) providing a pool of test agents;
(ii) determining whether any test agent from the pool inhibits the activity of at least one member selected from the group consisting of plasmin, plasminogen, urokinase-type plasminogen activator, urokinase-type plasminogen activator receptor, and plasminogen-activator inhibitor type 1, and
(iii) selecting any test agent from the pool that inhibits the activity of at least one member as an agent useful for treating or preventing arthritis.

17. The method of claim 16, which comprises a step of selecting the pool of test agents prior to step (i).

18. The method of claim 16, wherein the determining step comprises
(i) contacting a test agent from the pool with plasmin and a substrate to form a product;
(ii) measuring the level of the substrate or the product after the contacting step;
(iii) comparing the substrate level to a substrate control value or the product level to a product control value; and
(iv) selecting any test agent for which the substrate level is higher than the substrate control value or for which the product level is lower than the product control value as an agent useful in treating or preventing arthritis.

19. The method of claim 18, wherein the substrate is H-D-Valyl-L-leucyl-L-lysine-p-nitroaniline, and the product is p-nitroaniline dihydrochloride.

20. The method of claim 18, wherein the plasmin is formed by contacting plasminogen with a plasminogen activator, that is capable of promoting the formation of plasmin from plasminogen.

21. The method of claim 16, wherein the determining step comprises
(a) contacting a test agent from the pool with urokinase-type plasminogen activator and a substrate to form a product;
(b) measuring the level of the substrate or the product after the contacting step;
(c) comparing the substrate level to a substrate control value or the product level to a product control value; and
(d) selecting any test agent for which the substrate level is higher than the substrate control value or for which the product level is lower than the product control value as an agent that inhibits the urokinase-type plasminogen activator.

22. The method of claim 21, wherein the substrate is L-Pyroglutamyl-glycyl-L-arginine-p-Nitroaniline hydrochloride and the product is p-nitroaniline dihydrochloride.

23. The method of claim 16, wherein the determining step comprises
(a) contacting the test agent with urokinase-type plasminogen activator receptor and urokinase-type plasminogen activator;
(b) measuring the level of binding between the urokinase-type plasminogen activator receptor and urokinase-type plasminogen activator substrate after the contacting step;
(c) comparing the level of binding to a control value; and
(c) selecting any test agent for which the level of binding is lower than the control value as an agent useful for treating or preventing arthritis.

24. The method of claim 16, wherein the determining step comprises
(a) contacting the test agent with plasminogen-activator inhibitor type 1, an excess amount of urokinase-type plasminogen activator, and a substrate to form a product;
(b) measuring the level of the substrate or the product after the contacting step;
(c) comparing the substrate level to a substrate control value or the product level to a product control value; and
(d) selecting any test agent from the pool for which the substrate level is lower than the substrate control value or the product level is higher than the product control value as an agent capable of inhibiting the plasminogen-activator inhibitor type 1.

25. The method of claim 24, wherein the substrate is L-Pyroglutamyl-glycyl-L-arginine-p-Nitroaniline hydrochloride and the product is p-nitroaniline dihydrochloride.

26. The method of claim 16, wherein the test agent comprises an antigen-binding fragment of an antibody directed against the at least one protein.

27. The method of claim 16, further comprising selecting any test agent that inhibits the formation of plasmin from plasminogen as an agent useful in treating or preventing arthritis.

28. The method of claim 27, wherein the test agent inhibits the ability of urokinase-type plasminogen activator to promote formation of plasmin from plasminogen.

29. A method of identifying an agent that is useful in preventing or treating arthritis, which comprises:
(i) administering a test agent to a transgenic animal susceptible to collagen-induced arthritis, said animal lacking endogenous expression of at least one protein selected from the group consisting of plasmin, plasminogen, urokinase-type plasminogen activator, urokinase-type plasminogen activator receptor, and plasminogen-activator inhibitor type 1;
(ii) administering a human homolog of the at least one protein to the animal;
(iii) administering type II collagen to the animal to induce collagen induced arthritis in the animal;
(iv) determining the severity level of the induced collagen-induced arthritis in the animal;
(v) comparing the severity level to a control value; and
(vi) selecting any test agent for which the severity level is lower than the control value as an agent that is useful in preventing or treating arthritis.

30. The method of claim 29, wherein the control value is the severity level of collagen-induced arthritis in a control animal.

31. A method of identifying an agent as useful in treating rheumatoid arthritis which comprises administering a test agent to a mammal and determining whether the test agent inhibits plasmin, plasminogen, urokinase-type plasminogen activator, urokinase-type plasminogen activator receptor, or plasminogen-activator inhibitor type 1 in said mammal.

32. A method of identifying an agent that is useful in preventing or treating arthritis, which comprises:
(i) providing a pool of test agents;
(ii) mixing a test agent from the pool with plasmin and H-D-Valyl-L-leucyl-L-lysine-p-nitroaniline dihydrochloride, under conditions suitable for forming p-nitroaniline dihydrochloride from the H-D-Valyl-L-leucyl-L-lysine-p-nitroaniline dihydrochloride;
(iii) incubating the mixture for a predetermined time period;
(iv) measuring a test absorbance of the mixture at 405 nm;
(v) comparing the test absorbance with a control absorbance; and
(vi) selecting any test agent for which the test absorbance is lower than the control absorbance as an agent that is useful in treating or preventing arthritis.

33. The method of claim 32 wherein the predetermined time period is 4 hours, and the mixture is incubated at about 37° C.

34. The method of claim 32, wherein the control absorbance is the absorbance of a mixture of plasmin and H-D-Valyl-L-leucyl-L-lysine-p-nitroaniline dihydrochloride.

35. A method of identifying an agent that is useful in preventing or treating arthritis, which comprises:
(i) providing a pool of test agents;
(ii) mixing a test agent from the pool with plasminogen, an excess amount of urokinase-type plasminogen activator, and H-D-Valyl-L-leucyl-L-lysine-p-nitroaniline dihydrochloride, under conditions suitable for forming p-nitroaniline dihydrochloride from the H-D-Valyl-L-leucyl-L-lysine-p-nitroaniline dihydrochloride;
(iii) incubating the mixture for a predetermined time period;
(iv) measuring a test absorbance of the mixture at 405 nm;
(v) comparing the test absorbance with a control absorbance; and
(vi) selecting any test agent for which the test absorbance is lower than the control absorbance as an agent useful in treating or preventing arthritis.

36. The method of claim 35 wherein the predetermined time period is about 4 hours, and the mixture is incubated at about 37° C.

37. The method of claim 35, wherein the control absorbance is the absorbance of a mixture of plasminogen, an excess amount of urokinase-type plasminogen activator, and H-D-Valyl-L-leucyl-L-lysine-p-nitroaniline dihydrochloride.

38. A method of identifying an agent that is useful in preventing or treating arthritis, which comprises:
(i) providing a pool of test agents;
(ii) mixing a test agent from the pool with urokinase-type plasminogen activator and L-pyroglutamyl-glycyl-L-arginine-p-nitroaniline hydrochloride, under conditions suitable for forming p-nitroaniline dihydrochloride from the L-pyroglutamyl-glycyl-L-arginine-p-nitroaniline hydrochloride;
(iii) incubating the mixture for a predetermined time period;
(iv) measuring a test absorbance of the mixture at 405 nm;
(v) comparing the test absorbance with a control absorbance; and
(vi) selecting any test agent for which the test absorbance is lower than the control absorbance as an agent useful in treating or preventing arthritis.

39. The method of claim 38, wherein the predetermined time period is about 0.5 hours, and the mixture is incubated at about 37° C.

40. The method of claim 38, wherein the control absorbance is the absorbance of urokinase-type plasminogen activator and L-pyroglutamyl-glycyl-L-arginine-p-nitroaniline hydrochloride.

41. A method of identifying an agent that is useful in preventing or treating arthritis, which comprises:
(i) contacting a test agent with human urokinase-type plasminogen activator and an murine cell expressing a human urokinase-type plasminogen activator receptor, under conditions suitable for association of the human urokinase-type plasminogen activator to the receptor;
(ii) contacting the murine cell with a casein plaque; and
(iii) selecting any test agent for which the casein plaque is not degraded by the contacting in step (ii) as an agent useful in treating or preventing arthritis.

42. The method of claim 41 which comprises selecting the test agent from a plurality of test agents.

43. A method of identifying an agent that is useful in preventing or treating arthritis, which comprises:
(i) providing a pool of test agents;
(ii) mixing a test agent with plasminogen-activator inhibitor type 1, urokinase-type plasminogen activator and L-pyroglutamyl-glycyl-L-arginine-p-nitroaniline hydrochloride, under conditions suitable for forming p-nitroaniline dihydrochloride from the L-pyroglutamyl-glycyl-L-arginine-p-nitroaniline hydrochloride;
(iii) incubating the mixture for a predetermined time period;
(iv) measuring a test absorbance of the mixture at 405 nm;
(v) comparing the test absorbance with a control absorbance; and
(vi) selecting an agent useful in treating or preventing arthritis any test agent for which the test absorbance is higher than the control absorbance.

44. The method of claim 43 wherein the predetermined time period is about 0.5 hours, and the mixture is incubated at about 23° C.

45. The method of claim 43, wherein the control absorbance is the absorbance of plasminogen-activator inhibitor type 1, urokinase-type plasminogen activator and L-pyroglutamyl-glycyl-L-arginine-p-nitroaniline hydrochloride, incubated for 0.5 hours at 23° C.
